(19) United States
(12) Patent Application Publication
(10) Pub. No.: US 2023/0051381 A1
(43) Pub. Date: Feb. 16, 2023

(54) PHOTOCATALYTIC AIR PURIFICATION AND DISINFECTION COMPOSITION AND SYSTEM

(71) Applicant: CATALIFE LTD., Jerusalem (IL)

(72) Inventor: Hani GNAYEM, Baqa El-Gharbya (IL)

(21) Appl. No.: 17/963,073

(22) Filed: Oct. 10, 2022

Related U.S. Application Data
(63) Continuation-in-part of application No. PCT/IL2021/050420, filed on Apr. 13, 2021.
(60) Provisional application No. 63/008,891, filed on Apr. 13, 2020.

(51) Int. Cl. B01J 27/10; B01J 21/18; B01J 35/04; B01J 35/06; B01J 35/00; A61L 9/18

(52) U.S. Cl. CPC B01J 27/10; B01J 21/18; B01J 35/04; B01J 35/06; B01J 35/004; A61L 9/18; A61L 2209/14; A61L 2209/12; A61L 2209/22

(57) ABSTRACT

A combination of bismuth oxyhalides is provided, which is a photooxidant, antibacterial and antiviral. The combination of bismuth oxyhalides is added to a filter medium (e.g., a multistage filter) to decompose VOCs and/or eliminate bacteria and/or viruses. Suitable designs of multistage filters are also provided.

Compounds exhibiting photocatalytic activity are capable of accelerating oxidation reactions in response to light irradiation and are hence potentially useful in decomposing organic contaminants. The TiO₂ powder manufactured by Degussa Corporation (named P-25) is an example of a commercially available photocatalyst activated by ultraviolet light (UV).

A different class of compounds showing photocatalytic activity includes bismuth oxyhalides of the formula BiOHal, wherein Hal indicates a halogen atom, namely, BiOCl, BiOBr and BiOI, and mixed bismuth oxyhalides bearing two different halogen atoms, such as BiOCl_{y}Br_{1-y}.

We reported the synthesis of BiOCl_{y}Br_{1-y} (y>0.5) compounds, incorporation of Bi⁽⁰⁾ dopant into bismuth oxyhalides to enhance their photocatalytic action, their uses in eliminating contaminants from water and in conferring self-cleaning functionality to building materials such as gypsum (see Gnayem and Sasson in ACS Catalysis 3, p. 186-191 (2013); WO 2012/066545, WO 2015/019348 and WO 2016/125175). The photocatalytic activity of BiOCl_{y}Br_{1-y} (y>0.5) compounds and the Bi⁽⁰⁾-doped material was induced by visible light irradiation.

We have now found that certain bismuth oxyhalides can be combined to create strong photooxidative, antimicrobial and antiviral effects. Owing to its multifold action, the combination (which consists of two to four bismuth oxyhalides) can be integrated into an air filter, to enhance air-purification and air-disinfection in existing air-conditioning systems, e.g., installed in motor vehicles. We have also designed an air filter device in which the light source needed for activating the bismuth oxyhalides is placed internally within the filter in an efficient manner.

The inclusion of photocatalytic function in air filters, by addition of titanium oxide supported on a suitable layer, is known. For example, EP 960944, FIGS. 5 and 6, describe a basic design, which consists of TiO₂ air-permeable layer (2), and an air-permeable layer (3) for capturing floating particles, mounted in a frame (9). The light source—UV lamp for activating titanium dioxide—is external to the filter.

A different approach to the positioning of a light source, to activate photocatalytic coating applied onto a fabric web, is shown in US 2010/0029157. It involves warp and/or weft optical fibers woven with warp and weft binding yarns. UV radiation transmitted through the optical fibers is guided within the fabric web to activate the TiO₂ photocatalytic coating.

A useful overview of photocatalytic reactors for air purification is given by Ren et. Al [Journal of Hazardous Materials 325 (2017) 340-366]. The basic reactor configurations include:

(i) plate reactor, consisting of a plate coated with a photocatalyst layer and UV lamp positioned in parallel to the coated plate, with the air passing in the space between the plate and the lamp, perpendicularly to the light direction;

(ii) annular reactor, in which the inner cylindrical surface is coated with the photocatalyst and the UV lamp is positioned coaxially and concentrically inside the annular space through which the air is passed;

(iii) honeycomb monolith reactor, consisting of a perforated body coated with the photocatalyst and having a plurality of UV lamps attached to its surface; the air flows through the array of perforations arranged in honeycomb structure; and

(iv) fluidized bed reactor; the air flows upward through a bed consisting of the photocatalyst particles; externally positioned UV lamp illuminating onto the lateral surface of the reactor.

The main goals of the present invention are to replace the customarily used UV light-activated titanium dioxide with visible light-activated combination of bismuth oxyhalides (using light emitting diodes (LED), fluorescent and daylight); integrate the bismuth oxyhalides into various substrates, e.g., fibers, non-woven fabrics, textile products as well as aluminum and gypsum based matrices, in particular in substrates which can act as filter medium; and create a filter design in the form of thin layers incorporating the bismuth oxyhalides and an illumination array of LED sources. Owing to its compact structure, the filter of the invention is well suited for installation in a variety of air conditioning systems to decompose volatile organic contaminants and exert antimicrobial and antiviral action.

One aspect of the invention is a combination comprising at least two bismuth oxyhalide compounds selected from Groups A1, A2, A3 and B.

Group A1 includes Bi⁽⁰⁾ doped-bismuth oxyhalides. For example, Bi⁽⁰⁾ doped-BiOCl, Bi⁽⁰⁾ doped-BiOBr and especially Bi⁽⁰⁾-doped mixed bismuth oxyhalides, e.g., of the formula Bi⁽⁰⁾doped-BiOCl_{y}Br_{1-y}, with y≥0.5, e.g., 0.6≤y≤0.95, 0.7<y<0.95.

Group A2 includes mixed chloride-bromide bismuth oxyhalides in which chloride is the predominant halide, namely, of the formula BiOCl_{y}Br_{1-y}, with y≥0.5, e.g., 0.6≤y≤0.95, 0.7<y<0.95.

Group A3 includes single halide bismuth oxyhalides. That is, compounds of the formula BiOHal (Hal is chloride, bromide or iodide).

Group B includes bismuth oxyhalides of the formula BiOCl_{y}Br_{1-y}, with y<0.5, e.g., 0.1≤y≤0.4, 0.15≤y≤0.35. That is, in the Group B compounds, bromide is the major halide. Experimental results reported below indicate that bromide-rich BiOCl_{y}Br_{1-y} possess antimicrobial effect and are therefore of potential benefit in adding disinfecting component to air-conditioning systems.

Hereinafter we use the notation X/Y to indicate multiple bismuth oxyhalides combinations. For example, binary combination such as A1/A3, A1/B, etc. When two or more members of Group A are present in the combination, say, A1 and A2, then they are put inside square brackets, i.e., the following notation is used to indicate ternary combination consisting of A1, A2 and B: [A1+A2]/B. The total amount of Group A compounds is considered when expressing weight ratio relative to the Group B compound, and further mixing ratios of the elements of Group A members is also given.

One preferred combination of the invention comprises at least one member of the A group (at least one of A1, A2 and A3) and at least one Group B compound.

The [A]/B combination is proportioned by weight in the range 90:10 to 10:90, e.g., 80:20 to 20:80, preferably from 75:25 to 25:75. Some preferred multiple bismuth oxyhalides combinations for use in the present invention include:

A1/B, at mixing ratio in the range from 75:25 to 25:75. For example:

Bi⁽⁰⁾doped-BiOCl_{y}Br_{1-y}(0.7≤y≤0.95)/BiOCl_{y}Br_{1-y}(0.15≤y≤0.35).

[A1+A2]/B, at mixing ratio in the range from 75:25 to 25:75, while the mixing ratio A1:A2 is in the range from 7:1 to 1:1 (that is, A1 is predominant over A2). For example:

[Bi⁽⁰⁾doped-BiOCl_{y}Br_{1-y}(0.7≤y≤0.95)+BiOCl_{y}Br_{1-y}(0.7≤y≤0.95)]/BiOCl_{y}Br_{1-y}(0.15≤y≤0.35).

[A1+A3]/B, at mixing ratio in the range from 75:25 to 25:75, while the mixing ratio A1:A3 is in the range of 2:1 to 1:2. For example:

[Bi⁽⁰⁾doped-BiOCl_{y}Br_{1-y}(0.7≤y≤0.95)+BiOBr]/BiOCl_{y}Br_{1-y}(0.15≤y≤0.35).

Another preferred combination of the invention comprises Group A1 compound and Group A3 compound at mixing ratio in the range of 65:35 to 35:65. For example:

[Bi⁽⁰⁾doped-BiOCl_{y}Br_{1-y}(0.7≤y≤0.95)+BiOBr].

The preparation of bismuth oxyhalides is preferably based on the synthetic approach shown in our earlier publications WO 2012/066545 and WO 2015/019348, namely, dissolution of bismuth salt, such as bismuth nitrate, in water in an acidic environment supplied by an organic acid such as glacial acetic acid (the pH of the reaction mixture is preferably less than 4, and even more preferably less than 3.5, e.g., from 2.5 to 3, and more specifically around 3). The so-formed solution is then combined with appropriate molar amounts of halide sources. Either organic halides or inorganic halides can be used. Suitable organic halides are quaternary ammonium halide salts such as N⁺R₁R₂R₃R₄Cl⁻ and/or N⁺R₁R₂R₃R₄Br⁻, wherein R₁, R₂, R₃ and R₄ are alkyl groups, which may be the same or different. For example, organic halide sources which can be suitably used are selected from the group consisting of cetyltrimethylammonium bromide (abbreviated CTAB), cetyltrimethylammonium chloride (abbreviated CTAC), tetrabutylammonium chloride (abbreviated TBAC) and tetrabutylammonium bromide (abbreviated TBAB). Suitable inorganic halides are alkali halide salts such as sodium chloride, sodium bromide, potassium chloride and potassium bromide.

It should be noted that the use of quaternary ammonium halide salts leads to the formation of bismuth oxyhalide particles with flower-like surface morphology, whereas the use of alkali halide salts leads to the formation of bismuth oxyhalide particles with plate-like surface morphology. For the purposes of the present invention, either morphology is acceptable. Images recorded with scanning electron microscopy (SEM) indicate that the bismuth oxyhalide particles are largely spherical, in the form of microspheres exhibiting flower-like surface morphology. By the term “flower-like surface morphology” is meant that the spherical particles are characterized by the presence of individual thin sheets or plates arranged radially like petals, wherein two or more adjacent individual thin sheets are interconnected to form cells or channels which open onto the external surface of said spheres.

The general synthetic pathway described above can be adjusted to produce the bismuth oxyhalides of Group A (subgroups A1, A2 and A3) and Group B.

To prepare the Group A1 compounds, that is, Bi⁽⁰⁾ doped-bismuth oxyhalides, further information can be found in WO 2015/019348. Having combined the acidic solution of the bismuth salt and the halide sources, a reducing agent, e.g., hydride such as sodium borohydride, is added to the solution, to reduce the bismuth ion and form metallic bismuth as a dopant in the bismuth oxyhalide. The reaction favors the presence of ethanol to minimize foaming. The solid is recovered by filtration, washed and dried. Bi⁽⁰⁾ doping level varies from 0.1 to 7.0% (molar %; e.g., 0.1 to 5%, for example, from 1.0 to 3.0 molar %; the molar percentage of the dopant is calculated relative to the total amounts of the trivalent and zerovalent bismuth). The dopant is detectable with the aid of X-ray photoelectron emission spectroscopy (peak at 157±1 eV is assigned to metallic bismuth). Useful exemplary preparations can be found in Examples 5 to 8 of WO 2015/019348 and one illustrative preparation is given below (entitled “Preparation 1”).

To prepare the Group A2 compounds, that is, BiOCl_{y}Br_{1-y}, with y≥0.5, further details can be found in WO 2012/066545, particularly in Examples 1 to 5 of WO 2012/066545. One illustrative preparation is given below (entitled “Preparation 2”).

To prepare the Group A3 compounds, the synthesis described in WO 2012/066545 can be used, particularly the chemical procedures under “Preparation 1” and “Preparation 2” which are found at the experimental section of WO 2012/066545. We have observed that the synthesis of BiOBr by dissolution of bismuth source (e.g., the nitrate salt) in an aqueous mixture of acetic acid, followed by addition of quaternary ammonium salt, affords BiOBr particles with the flower-like morphology which exhibit antiviral activity. One illustrative preparation is given below (entitled “Preparation 3”).

For the preparation of Group B bismuth oxyhalides, namely, BiOCl_{y}Br_{1-y}, with y<0.5, the synthesis described in WO 2012/066545 can be used, with appropriate adjustment of the molar amounts of the chloride/bromide sources, to reverse the halide predominance. As to the chloride/bromide sources, the invention contemplates either the use of alkali halides or organic halides. BiOCl_{y}Br_{1-y}, with y<0.5, e.g., 0.15≤y≤0.35, are eventually recovered as particles with plate-like surface morphology, or flower-like surface morphology, depending on the halide source. Illustrative preparations are given below (entitled “Preparation 4” and “Preparation 5”, using alkali halides and quaternary ammonium halides, respectively). The former preparation affords BiOCl_{y}Br_{1-y} [y<0.5] showing photooxidation action (i.e., elimination of volatile organic compounds), whereas the latter preparation leads to BiOCl_{y}Br_{1-y} [y<0.5] exhibiting in addition biological activity.

The bismuth oxyhalide for use in the invention are crystalline, as demonstrated by their X-ray powder diffraction patterns. For example, bismuth oxychloride exhibits characteristic peaks at 12.02 2θ±0.05 and one or more peaks at 26.01, 32.25, 40.82 and 58.73 2θ (±0.05 2θ). Bismuth oxybromide exhibits characteristic peaks at 11.0 2θ±0.05 and one or more peaks at 31.78, 32.31, 39.26, 46.31, 57.23, 67.53 2θ±0.05. The mixed BiOCl_{y}Br_{1-y} compounds of the invention exhibit X-ray powder diffraction pattern having a characteristic peak in the range from 11.0 to 12.2 2θ (±0.05 2θ), which peak is indicative of the Cl:Br ratio. In other words, the exact position of the indicative peak within the 11.0-12.2 2θ interval depends essentially linearly on the Cl:Br ratio, as predicted by the Vegard rule. The chemical composition of the compound belonging to the family BiOCl_{y}Br_{1-y} wherein y is as defined above can be determined using EDS analysis. The composition of the BiOCl_{y}Br_{1-y} compound can be also determined using XRD data and Vegard's law.

Particle size measured with Malvern Instruments—Mastersizer 2000 particle size analyzer shows that the average diameter of the bismuth oxyhalides particles is from 2 to 5 microns, more specifically from 3 to 4 microns.

The preferred compounds for use in the invention have surface area of not less 8 m²/g, more preferably not less than 30 m²/g, as determined by BET (the nitrogen adsorption technique). The BET surface area of the subgroup A1 compounds, subgroup A2 compounds, subgroup A3 compounds and Group B compounds is generally from 8 to 80 m²/g, respectively.

The properties of the A1, A2, A3 and B compounds that are demonstrated by the experimental work conducted in support of this invention are tabulated below, such that suitable combinations can be selected to meet specific needs:

| | photooxidation | | |
|---|---|---|---|
| | (VOCs and/or NOx); | | |
| | imparting ‘self- | | |
| | cleaning’ function | Antibacterial | Antiviral |
| Compound | to a substrate | action | action |
| A1 (quaternary | √ | √ | √ |
| ammonium halides | | | |
| are used in synthesis) | | | |
| A2 (quaternary | √ | | |
| ammonium halides | | | |
| are used in synthesis) | | | |
| A3 (quaternary | | | √ |
| ammonium halides | | | |
| are used in synthesis) | | | |
| B (alkali halides | √ | | |
| are used in synthesis) | | | |
| B (quaternary | √ | √ | √ |
| ammonium halides | | | |
| are used in synthesis) | | | |

The A/B and [A₁+A₃] powder blends can be used for air purification/disinfection in different ways. For example, the A/B and [A₁+A₃] powder blends can be applied on surfaces in photocatalytic reactors for air purification, according to configurations described above. That is, in a plate reactor (to coat the surface of a plate), in an annular reactor (to coat the inner walls of a cylindrical reactor) and fluidized bed reactor (to be packed and serve as the bed).

In the reactor configuration set out above, air purification is achieved when the air flow passes in parallel to the bismuth oxyhalides-coated surfaces or through a bed consisting of bismuth oxyhalides particles. Additionally, experimental results reported below indicate that the A/B and [A₁+A₃] combinations fit well into applications involving the use of air-preamble substrates designed to permit passage of air therethrough. That is, when the air flows across bismuth oxyhalides-added porous or fibrous substrates or structures, akin to the air-purification approach implemented in the honeycomb monolith reactor mentioned above. Examples of air-preamble substrates which could benefit from incorporation (e.g., by coating, impregnation etc.) of the bismuth oxyhalides combination include fabrics (e.g., nonwoven fabrics and other textile products) as well as porous aluminum and gypsum-based matrices. For example, an air-permeable substrate could have a design of an array of hollow cells formed between thin walls (0.5-4 mm thick) made of a metal (e.g., aluminum) or gypsum; resembling honeycomb structure when the cells are hexagonal in shape, to which the bismuth oxyhalides are added by the methods illustrated below. When the air moves across the hollow cells, it is exposed to the action of the photocatalysts.

Bismuth oxyhalides-added air-permeable substrate configured to enable passage of air across the substrate, when used as an air filter component (e.g., installed in cabin air filters, in air conditioning apparatuses used domestically, at hospitals or in clean rooms, in medical masks, to name a few examples), achieve, under visible light illumination, air purification and disinfection effects by oxidation of volatile organic contaminants (VOC) and elimination of bacteria (gram negative, gram positive) and viruses, as shown by the experimental work reported below. The experimental set-up consisted of a 500 L sealable test chamber, in which a sample of a volatile organic solvent was evaporated. The interior air (bearing few ppm's of the organic vapors) was forced to circulate through a 3 L rectangular photocatalytic cell (10 cm×10 cm×30 cm) placed in the test chamber. The bismuth oxyhalides-added air-permeable substrate was fixed inside the photocatalytic cell, 10 cm apart from a visible light lamp placed inside the cell, parallel to the substrate. An array of fans sets the air in motion across the visible light-activated photocatalytic cell.

In separate studies, the same photocatalytic cell was used to measure the antimicrobial effect exerted by the bismuth oxyhalides-added air-permeable substrate.

For example, we tested the efficiency of bismuth oxyhalides-added air-permeable gypsum, using a square prism-shaped gypsum body perforated by an array of open cells arranged in honeycomb structure, to enable air flow across the gypsum block, that is, through passages with hexagonal cross section. The combinations of bismuth oxyhalides demonstrated strong photooxidation and antimicrobial action under visible light irradiation, achieving mineralization of vapors of organic contaminants forced through the perforated gypsum mold and elimination of surface bacteria.

We also tested the efficiency of bismuth oxyhalides-added air-permeable nonwoven fabric using the same experimental set-up, that is, replacing the honeycomb-shaped gypsum photocatalytic filter with nonwoven fabric which was spray coated with ethanolic dispersion of bismuth oxyhalides, dried and mounted in the photocatalytic cell. The bismuth oxyhalides-added air-permeable nonwoven fabric displayed strong photocatalytic oxidation activity.

Accordingly, another aspect of the invention is a filter medium comprising bismuth oxyhalides added to a flow-through support.

By “flow-through support” is meant an air-permeable substrate configured to enable passage of air across the substrate. Preferably, the substrate is selected from the group consisting of fabrics (for example, woven or nonwoven fabrics made of natural or synthetic fibers (cotton, polyester, polyamide, polypropylene, carbon, silica, glass). The loading of the bismuth oxyhalides, expressed as weight % relative to the substrate (e.g., fabric) weight is in the range from 1 to 10% (or, expressed otherwise, from 0.01 g/cm² to 0.10 g/cm²).

Various techniques can be used to integrate the bismuth oxyhalides in the flow-through support, such as spreading, coating (spray coating, spin coating, electrospinning), padding (pad-dry-cure), dipping and printing.

For example, the bismuth oxyhalides powder blend (proportioned as appropriate) can be added to water, a volatile organic carrier, e.g., ethanol, or a mixture thereof, optionally in the presence of one or more binders [sodium silicate, sodium aluminate (or their mixture with poly(vinyl alcohol)), alumina, silica, styrene acrylic] and functional additives [activated carbon, graphite, which act as adsorbents as discussed below) or other types of additives (for example, capping agent such as PVA) to form an aqueous or ethanolic dispersion to be applied onto suitable substrates. Therefore, a composition comprising the bismuth oxyhalides combination of the invention (e.g., A1+B, A1+A2+B, A1+A3, A1+A3+B) in a liquid carrier, that is, water, volatile organic solvent (e.g., ethanol) or aqueous/volatile organic medium (e.g., water:EtOH mixture with ethanol content from 5 to 30% by volume), wherein the concentration of the bismuth oxyhalides is from 0.5 to 25 wt. % based on the total weight of the composition—either binder-free or added-binder composition—forms additional aspect of the invention.

One method of binding the bismuth oxyhalides to a flow-through support, e.g., fabric support, is by spray coating. We achieved good results by formulating binder-free sprayable ethanolic dispersions of bismuth oxyhalide powder blends, spraying them to coat support fabrics, and drying the fabrics, under ambient temperature to remove the volatile carrier. The physical (no-binder) entrapment of the photocatalytic mixtures inside the porous structure of the coated fabric has shown to be satisfactory.

Another approach to the fusion of the bismuth oxyhalides in a flow-through support is to force the bismuth oxyhalide formation reaction to take place in the porosity of the support, namely, in-situ generation of bismuth oxyhalides-embedded fabric. An aqueous or organic solution of the bismuth salt is added to a photocatalyst flow-through support (NWF, fiber/cloth) containing the same pore volume as the volume of the solution that was added. Capillary action draws the solution into the pores. Next, halides solution Is sprayed to complete the in-situ generation and deposition of the photocatalysts onto the fibers.

Adhesives available in the marketplace could also be used to attach the bismuth oxyhalide particles to the flow-through support. Suitable elastomeric binders are formulated in organic solvents in a sprayable form. With optional dilution, these formulations can be used to coat the flow-through support. After the organic volatile components evaporate, the bismuth oxyhalides (as a powder, or in an organic dispersion, e.g., C2-C3 alcohol) are applied onto the glue-coated surface.

Additionally, the invention provides a filter medium comprising bismuth oxyhalides added to a flow-through support that is made of gypsum. For example, a square prism-shaped gypsum body perforated by an array of open cells arranged in honeycomb structure (or other shapes, of course), to provide passages extending across the gypsum body, through which the air can flow. The gypsum body can be formed, with bismuth oxyhalides deposited onto its surface and walls (i.e., the inner walls of the passages extending across the gypsum), with the aid of a suitable template. For example, a silicon made-template consisting of an array of silicon-made hexagonal prisms extending vertically from a silicon base, as shown in more detail below.

One way to produce a gypsum-based filter with the aid of such template is through direct mixing of the bismuth oxyhalides (in a fine powder form) with the gypsum powder in a suitable volume of water, following which the resulting mixture (gypsum-water-photocatalyst) is poured into the template. In this way, and while using a proper weight ratio of the photocatalyst (5 to 40 wt. % relative to the gypsum powder weight), one will get the final structure with a high loading of the photocatalyst on the surface. Small amounts of adsorbents, e.g., activated carbon and silica (each 1 to 10 wt. % relative to the gypsum powder weight) can be added to mixture.

Another useful method to form a gypsum-based filter is to apply onto the template an aqueous dispersion of the bismuth oxyhalides (e.g., by brushing or spraying). The next step is to pour a freshly prepared gypsum into the template. This will lead to the adsorption of the photocatalyst on the top layers of the resulting gypsum structure. Final curing/hardening time is few hours (typical to gypsum).

The invention further provides a filter medium comprising bismuth oxyhalides added to a flow-through support that is made of a metal, e.g., aluminum. Again, an exemplary design consists of the closely spaced hollow cells defined by thin aluminum walls, e.g., in honeycomb structure. To apply the bismuth oxyhalides onto the aluminum walls, precoating with the abovementioned elastomeric binders [formulated in an organic volatile vehicle, e.g., ˜ 20-30% by weight solid content; viscosity in the order of a few hundreds centipoise] is needed.

The bismuth oxyhalides-added flow-through supports described above could be integrated in filtration devices to maintain good automobile interior air, planes and vessels interior air or good indoor air quality at home, refrigerators, elevators, office buildings and hospitals.

For example, experimental results shown below suggest that bismuth oxyhalides could greatly improve the performance of filters based on activated carbon—a commonly used adsorbent in many filters. The major function served by activated carbon is the removal of particulate matter and odors, for example, as a pre-filter component is multistage filter. Work conducted in support of this invention shows that enhanced rate of removal/decomposition of volatile organic contaminates is achieved by admixing bismuth oxyhalides and activated carbon, combining the adsorption action of the carbon particles with the strong oxidation and antimicrobial activity of the bismuth oxyhalides. Blends of activated carbon and bismuth oxyhalides therefore constitute another aspect of the invention, such as 10:90 to 90:10, e.g., 70:30 to 30:70, proportioned by weight. A filter medium comprising bismuth oxyhalides in admixture with activate carbon applied on a flow-through support is also provided by the invention.

A specific aspect of the invention relates to a multistage filter comprising VOC-decomposing and/or bacteria-eliminating and/or viruses-eliminating filter medium in the form of photocatalyst supported on a flow-through layer, placed downstream to a prefilter, with light source positioned between said prefilter and said photocatalyst, such that said light source faces said photocatalyst.

More specifically, the multistage filter comprises bismuth oxyhalides supported on a flow-through layer, optionally in admixture with activated carbon, wherein said flow-through layer is disposed between a pre-filter layer and a post-filter layer, wherein said pre-filter and post-filter layers are particulate-trapping layers, and wherein the light source consists of a plurality of LED lamps illuminating the photocatalyst.

A cabin air filter is just one example that springs to mind, which could benefit from the multistage filter design. Studies show that air quality inside the vehicle is 6× to 12× worse than outside. Inhalation exposure to VOCs during an 80 min drive is approximately equivalent to that of staying at a home for 16.5 h. (ratio of material volume to space volume in vehicles).

A cabin air filter consists of pleated fibrous material, functioning to maintain a steady stream of clean air flowing into the car. Before entering the interior of the vehicle, namely, the driver and passengers compartment, outside air passes through the filter to capture the contaminants inside the filter.

One illustrative design for an air filter of the invention is shown in FIGS. 14-15 (exploded-side views). The bismuth oxyhalides added-flow-through support layer (L2) is disposed between a pre-filter layer (L1) and a post-filter layer (L3), capable of capturing particulate matter (L1 removes airborne particles carried by the incoming outside air, whereas L3 prevents bismuth oxyhalides particles that may be detached from L2 from entering the passengers compartment). The air filter of the invention may be either pleated air filter, as shown in FIGS. 14-15, or non-pleated. The layers L1, L2 and L3 correspond in shape and size, and fit into square or rectangular frame (not shown), installable in the ventilation system of a vehicle (where a fan draws outside air stream and forces it through the filter to the interior of the vehicle).

In the embodiment shown in FIGS. 14 and 15, the light source needed for activating the photocatalysts is located internally inside the cabin air filter. The light source could be in the form of illumination array consisting of evenly distributed LED (for example, ˜10 W power blue LED lamps). For example, an array consisting of LED chains extending parallel to one another, for example, 1-2 cm apart from one another, to supply uniform illumination, e.g., irradiation density of 0.5-10 mW/cm² or specifically 1-7 mW/cm², could be attached to the pleats of either the L1 or L2 layers. Another way to integrate LED lamps inside an air cabin filter is with the aid of a scaffold, for example, aluminum-made rectangular frame with thin wires extending in parallel from one side of the frame to the opposite side (“illumination array” shown in FIG. 15), to correspond in shape, size and position to the pleated structure of L2. Such scaffold, with the LED lamps supported thereon, could be placed 1 to 5 mm apart from the bismuth oxyhalides-added layer L2, and the plurality of layers could be stacked together to form a compact cabin air filter structure.

The cabin air filter described in detail above is just one example of a device utilizing the filter medium of the invention. Such air filter medium, with the bismuth oxyhalides applied on a flow-through support, can be provided in different shapes and dimensions and may be mounted in a suitable housing to permit passage of air therethrough, or may fit into the air flowing area of an air tube or an air conditioning system. A suitable design is shown in FIG. 22.

The direction of the incoming air flow in air tube (20) is indicated by an arrow. The components of the multistage filter (21) are circular in shape (to match the cross-section of the tube) and include a prefilter layer, e.g. HEPA (22), flow-through supports, to which the combination of photocatalyst was added (23A and 23B); arrays of LED lights (24A and 24B) to illuminate the adjacent photocatalysts-added flow-through supports (23A and 23B), for example, at night time or when daylight is insufficient, and a postfilter (25) positioned downstream. In FIG. 22, the individual components are distanced apart from each other for the purpose of illustration; in use, the components are stacked.

Owing to the activatability of the bismuth oxyhalides in response to daylight illumination, a housing accommodating the filter medium would be made, at least in part, of transparent walls.

Another aspect of the invention is a transparent photocatalytic cell, having an air inlet and an air outlet, comprising:

bismuth oxyhalides-added filter medium mounted inside the cell;

means for drawing outside air stream or circulated air stream into the cell and forcing said air stream across said filter medium,

wherein the bismuth oxyhalides photocatalyst is activatable by daylight entering the cell or visible light source positioned to illuminate said photocatalyst.

The transparent photocatalytic cell may have a rectangular or cylindrical shape, with the filter medium positioned perpendicularly to the longitudinal or axial direction of the rectangular or cylindrical cell, respectively, to occupy the cross-section area of the cell. For example, the cell has a front face, which is perforated, permitting passage of air, and a rear face, adjacent to which a fan or a blower is placed to draw outside air stream into the cell. Such photocatalytic cell could be part of a portable or a fixed device. To avoid interruption in the operation of the cell due to insufficient daylight influx, a visible light source, such as White LED lamp (e.g., 6500K with optional 10-40 W power) can be placed inside the cell to effectively illuminate the photocatalyst.

The photocatalytic cell can be installed in air flowing areas of air tubes, air tunnels or air conditioning systems, that are exposed to daylight, e.g., in parts placed on a roof of buildings (e.g., hospitals). For example, a secondary air stream, drawn from a major air flow line, may be guided through secondary air flow line diverging from, and returning to, the major air flow line, with the photocatalytic cell being located along said secondary air flow line.

Spaces prone to development of surface microbial contamination, such as hospital rooms, food production and/or storage plants, could benefit from the present invention owing to the antibacterial and antiviral activity of the bromide-rich mixed bismuth oxyhalide (Group B compounds) or Group A3 bismuth oxybromide. The experimental results reported below demonstrate that reduction of microbial load on surfaces could be achieved without direct contact between the filter medium of the invention and the contaminated surfaces. It is assumed that air stream that passes through the filter becomes progressively loaded with oxidant species, enabling the photocatalyst to exert remote antimicrobial effect reaching the contaminated surfaces.

Accordingly, another aspect of the invention is a method for reducing microbial (bacterial, viral) load on surfaces, comprising forcing the air in a space where the surfaces are placed to pass across a filter medium having a combination of bismuth oxyhalides applied on flow-through support, wherein said bismuth oxyhalides include bromide-predominant mixed halide of the formula BiOCl_{y}Br_{1-y}, with y<0.5, said bismuth oxyhalides being illuminated by visible light (to load the air with oxidant species and reduce the level of microorganism on said surfaces without the direct application of oxidant species onto said surfaces).

### IN THE DRAWINGS

FIG. 1 is SEM image of BiOCl_{0.80}Br_{0.20} microspheres.

FIG. 2A is SEM image of BiOCl_{0.20}Br_{0.80} plates.

FIG. 2B is SEM image of BiOCl_{0.20}Br_{0.80} microspheres.

FIG. 3 is a photo of a gypsum-made honeycomb-shaped filter.

FIG. 4 is a photo of a silicon template used to create the gypsum-made honeycomb-shaped filter.

FIGS. 5A and 5B illustrate the design of a photoreactor.

FIG. 6 illustrate the design of a cell housing a volatile solvent and the photoreactor placed in the cell.

FIG. 7 shows VOC (toluene) concentration versus time plots.

FIG. 8 shows VOC (ethanol) concentration versus time plot.

FIG. 9 is a photo of the experimental set-up used for the biological study.

FIG. 10 is a photo of air cabin filter.

FIG. 11 is toluene concentration versus time plot.

FIGS. 12A and 12B demonstrate the combined action of activated carbon and the photocatalysts of the invention.

FIG. 13 is formaldehyde concentration versus time plot.

FIG. 14 is a photo showing a multistage filter.

FIG. 15 illustrates a multistage filter with the illumination array incorporated therein.

FIG. 16 is toluene concentration versus time plot.

FIG. 17 is toluene concentration versus time plot.

FIG. 18 illustrates an experimental set-up of the photoreactor.

FIG. 19 is a photo showing a series of aluminum flow-through supports.

FIG. 20 is toluene concentration versus time plot.

FIG. 21 is toluene concentration versus time plot.

FIG. 22 shows the incorporation of a multistage filter inside air tube or air channel.

FIGS. 23A, B and C. FIG. 23A is a graph showing the virus infectivity assay (end-point dilution) performed as described in Example 8 for cells infected with viruses pre-treated with the compound Bi⁽⁰⁾ doped-BiOCl_{0.80}Br_{0.20} for 5 minutes in the light or for 25 minutes in the dark. Virus titers at 5 minutes (indicated in the graph by the label “Catalife Light”) and 25 minutes (indicated in the graph by the label “Catalife Dark”) are presented here as 1 IU/ml, while in fact the CPE was absent in all the dilutions tested. Vero-E6 cells in 96-well plates were fixed and stained with Crystal Violet 48 hours post infection in the absence (“Mock”, B) or in the presence of the compound (Bi⁽⁰⁾ doped-BiOCl_{0.80}Br_{0.20}, C). Blue-stained wells indicate no CPE detected, while empty (“white”) wells indicate CPE. Several dilutions of the virus out of 10⁻² to 10⁻⁷ are presented. Triplicates are presented here for the virus dilution 10⁻³ and 10⁻⁴.

### EXAMPLES

### Preparation 1

### Preparation of Component A1: Bi⁽⁰⁾ doped-BiOCl_{0.80}Br_{0.20}

Deionized water (75 ml), glacial acetic acid (35 ml) and bismuth nitrate (9.18 g) were added to a flask and were mixed at room temperature for fifteen minutes until a clear, transparent solution was formed. The so-formed solution was added to a previously prepared solution consisting of CTAC (4.85 g; in the form of 25 wt % aqueous solution) and CTAB (1.378 g dissolved in 10 ml of water). Finally, sodium borohydride (21.4781 mg) and ethanol (20 ml) were added to the reaction mixture, which was then stirred for additional 60 minutes at about 25-30° C. The precipitate formed was separated from the liquid phase by filtration, washed five times with ethanol (5×50 ml) and then five times with water (5×200 ml). The off-white solid was then dried (3 hours in air). The weight of the solid collected was ˜9 grams. Doping level was ˜3%.

### Preparation 2

### Preparation of Component A2: BiOCl_{0.80}Br_{0.20}

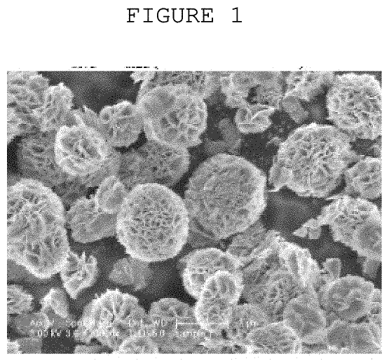

Deionized water (75 ml), glacial acetic acid (35 ml) and bismuth nitrate (9.18 g) were added to a flask and mixed at room temperature for fifteen minutes until a clear, transparent solution was formed. CTAB (1.378 g dissolved in 10 ml of water) and CTAC (4.85 g; in the form of 25 wt % aqueous solution) were added to the solution, for additional 30 minutes of mixing at room temperature. The white precipitate formed was separated from the liquid phase by filtration, washed five times with ethanol and five times with water, in order to remove the non-reactive organic species. The solid was then dried (in air). The weight of the solid collected was 7 g. The product may be subjected to heating at 400° C. for approximately 1 hour. The entitled product is characterized by average particle size of 2.62 m, surface area of 25.75 m²/g and pore radius of 22 Å. As shown in FIG. 1, the so-formed BiOCl_{0.80}Br_{0.20} has flower-like morphology.

### Preparation 3

### Preparation of Component A3: BiOBr

Deionized water (50 ml), glacial acetic acid (35 ml) and bismuth nitrate (9.70 g) were added into a 250 ml beaker and mixed at room temperature for fifteen minutes until a clear, transparent solution was formed. Cetyltrimethylammonium bromide-CTAB- (7.2879 g dissolved in 30 ml of water and 30 ml of ethanol) were added to the solution, for additional 60 minutes of mixing at room temperature. The yellowish precipitate thus formed was separated from the liquid phase by filtration, washed five times with water (50 ml) and washed five times with ethanol (30 ml), in order to remove the non-reactive species. The solid was then dried (in air or oven at 60° C./overnight).

### Preparation 4

### Preparation of Component B: BiOCl_{0.20}Br_{0.80} (Using Inorganic Halides)

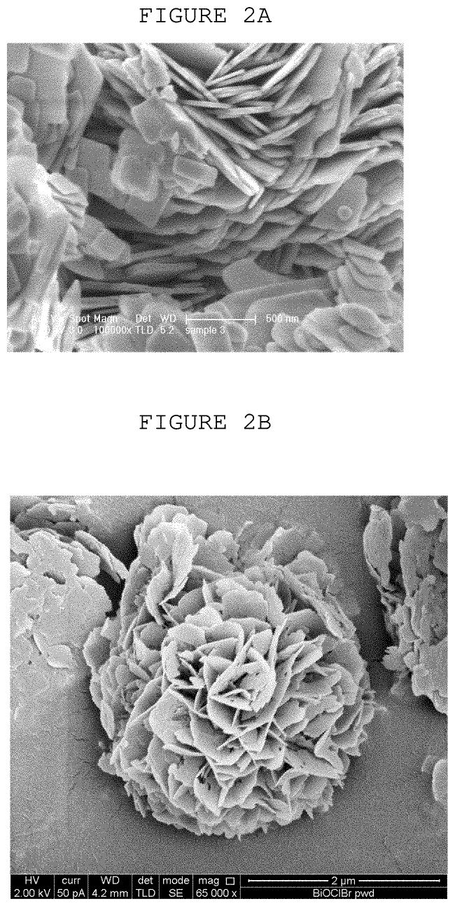

Deionized water (50 ml), glacial acetic acid (35 ml) and bismuth nitrate (9.18 g) were added into a 250 mL beaker and mixed at room temperature for fifteen minutes until a clear, transparent solution was formed. Sodium chloride-NaCl (0.2212 g dissolved in 10 ml of water) and potassium bromide-KBr (1.8017 g dissolved in 10 ml of water) were added to the solution, for additional 60 minutes of mixing at room temperature. The yellowish precipitate thus formed was separated from the liquid phase by filtration, washed five times with water (50 ml), in order to remove the non-reactive species. The solid was then dried (in air or oven at 60° C./overnight). The entitled product was characterized by average particle size of 7 μm, BET surface area of about 30 m²/g and pore radius of 22 Å. As shown in FIG. 2A, the so-formed BiOCl_{0.20}Br_{0.80} has plate-like morphology.

### Preparation 5

### Preparation of Component B: BiOCl_{0.20}Br_{0.80} (Using Organic Halides)

Deionized water (50 ml), glacial acetic acid (35 ml) and bismuth nitrate (9.18 g) were added into a 250 ml beaker and mixed at room temperature for fifteen minutes until a clear, transparent solution was formed. Cetyltrimethylammonium chloride-CTAC- (4.8448 g of 25 wt. % aqueous solution) and Cetyltrimethylammonium bromide-CTAB- (5.5178 g dissolved in 20 ml of water and 15 ml of EtOH) were added to the solution, for additional 60 minutes of mixing at room temperature. The yellowish precipitate thus formed was separated from the liquid phase by filtration, washed five times with ethanol (30 ml) and five times with water (50 ml), in order to remove the non-reactive organic species. The solid was then dried (in air or oven at 60° C./Overnight). As shown in FIG. 2B, the so-formed BiOCl_{0.20}Br_{0.80} has flower-like morphology.

### Example 1

### Photooxidation Activity of [A1+A2]/B Combination Incorporated into Gypsum Model Filter: Decomposition of Toluene and Ethanol

The goal of the study was to determine the visible-light induced photooxidation generated by a combination of bismuth oxyhalides, to assess its potential benefit in air-purification, i.e., in decomposing volatile contaminants.

In the study reported in this Example, a combination of three active bismuth oxyhalides was formulated as an aqueous dispersion. The formulation was applied onto honeycomb-shaped, gypsum-made filter. The photocatalytic filter was mounted in a cell, equipped with visible light irradiation source (to “switch on” the photocatalytic activity) and a fan. Vapors of volatile organic solvents, generated in a sealed test chamber, were caused to flow through the cell and across the photocatalytic filter. The concentration of the gaseous organic material was measured as function of time for more than 10 hours, to assess the ability of the photocatalytic filter to decompose vapors of organic contaminants passing therethrough.

Experimental Set-Up

1) A1+A2+B Aqueous Formulation

30 g of component A (Bi⁽⁰⁾ doped-BiOCl_{0.80}Br_{0.20} of Preparation 1), 10 g of component A2 (BiOCl_{0.80}Br_{0.20} of Preparation 2) and 20 g of component B (BiOCl_{0.20}Br_{0.80} of Preparation 4) are added to 100 ml water, to afford an aqueous dispersion of the three photocatalysts.

2) Photocatalytic Filter

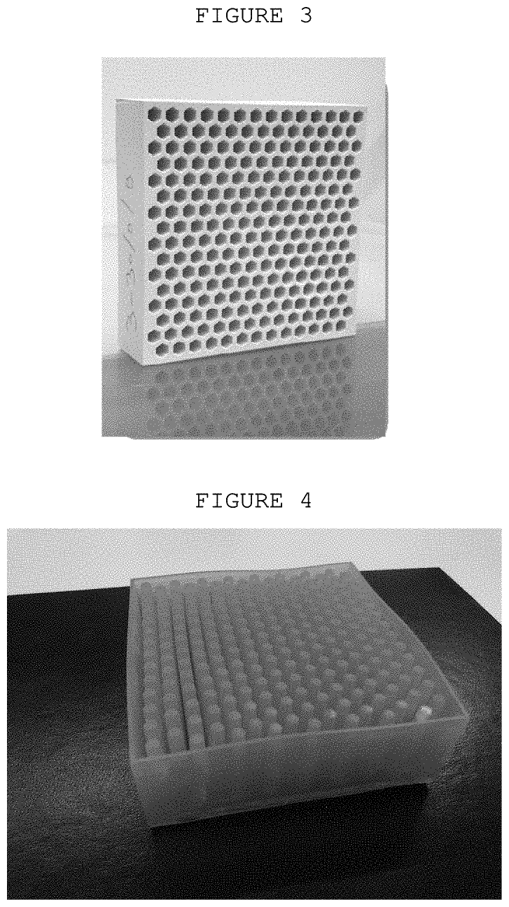

The filter model is made of gypsum body shaped into a square prism (a=10 cm, b=10 cm, c=3 cm), as shown in FIG. 3, with an array of open cells arranged in honeycomb structure, to allow air flow across the gypsum block, that is, through the passages extending perpendicularly to the bases (note that each passage has hexagonal cross section).

The rectangular gypsum block is prepared with the aid of a corresponding template shown in FIG. 4. The open cells in the honeycomb-shaped gypsum filter of FIG. 3 correspond in shape, size and position to the hexagonal prisms of the template shown in FIG. 4. The template consists of an array of 216 silicon-made hexagonal prisms extending vertically from a silicon frame. Each hexagonal prism is 3.5 cm high; the side of the hexagonal base is 5 mm. The center-to-center distance between two adjacent hexagonal prisms in a row is 5 mm.

Gypsum powder (180 g) was added to the A1+A2+B aqueous dispersion, and the so-formed mixture was poured into the silicon template. The hardening process of the gypsum took a few hours, following which the photocatalysts-added gypsum filter was ready for use.

3) Photocatalytic Reactor

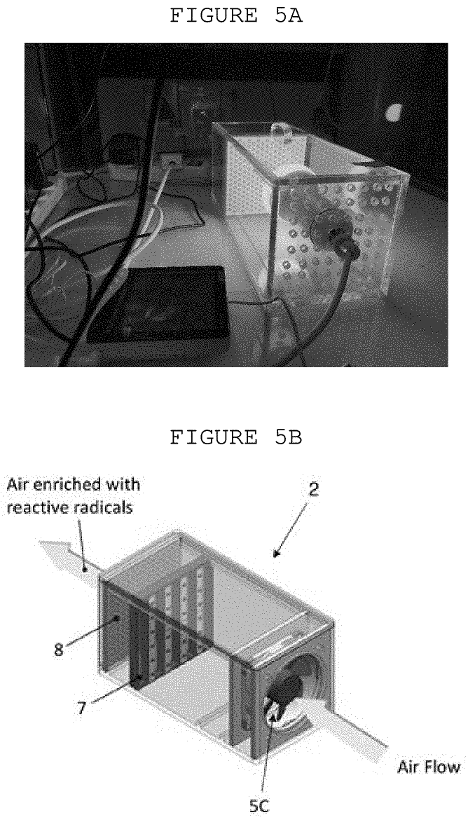

The photocatalytic reactor is shown in the photo appended in FIG. 5A. It consists of a Perspex cell (length: 30 cm, width: 10 cm, height: 10 cm). The walls of the cell are 5 mm thick. The honeycomb-shaped gypsum cast was placed at distance of 10 cm from, and parallel to, one of the square faces of the Perspex cell. White LED lamp (6500K with optional 10-40 W power) extends from the opposite square face of the cell into the interior of cell, illuminating in the direction of the gypsum body. The distance between the gypsum cast and the lamp was about 10 cm. Air flow across the cell was aided by a fan mounted on one face of the cell (in the side of the gypsum filter) and apertures distributed over the opposite face of the cell (where the lamp is placed).

4) Test Chamber

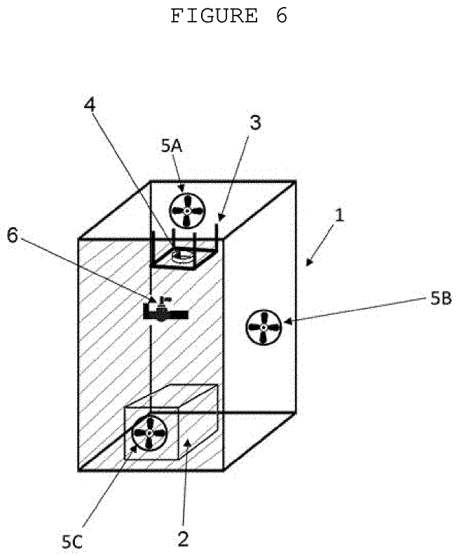

The test chamber, which is shown in FIG. 6, consists of 500 L sealable cell, rectangular in shape (1) designed to accommodate the photocatalytic reactor and allow a flow of vaporized organic pollutant across the photocatalytic reactor (2), and measurement of the concentration of the gaseous pollutant in order to determine the degree of decomposition that can be achieved with the aid of photocatalytic reactor.

A shelf (3) is mounted at the upper part of the test chamber. The purpose of the shelf is to hold a petri dish (4), which is filled with the tested volatile organic solvent. The test chamber was equipped with a pair of fans (5A and 5B), one located above the shelf, to facilitate the vaporization of the organic solvent. The other fan (5B) is located on one of the walls of the test chamber, to ensure effective distribution of the vaporized organic pollutant in the interior of test chamber and passage of the vapors through the photocatalytic reactor. The photoreactor (2) is equipped with its own fan (5C), as previously explained. The test chamber is provided with a sealable door (not shown).

The test chamber also includes an external tap (6) mounted in the center of one of its walls, where VOC measurement occurs. The test chamber is equipped with a humidity and a temperature meter. The gas concentration in the test chamber was measured using Tiger VOC detector (from Ion Science), a photoionization detector equipped with 10.6 eV ionization lamp which measures concentrations of a wide range of gases, from 20,000 ppm down to 1 ppb.

Experimental Protocol

The test chamber was ventilated before the experiment begun to ensure that the atmosphere inside the test chamber was the same as the outside atmosphere. This atmosphere was set as the zero point for the measurements of the Tiger photoionization detector, such that any reading of the detector was relative to the zero point.

A petri dish with a sample of the tested organic solvent was placed on the shelf in the test chamber and the chamber was sealed. The pair of fans inside the test chamber were turned on and allowed to operate for thirty minutes. During the thirty minutes time period, the photocatalytic reactor placed in the cell is inactive: neither the fan nor the lamp of the photocatalytic reactor was switched on. Meanwhile the vapors of the slowly evaporating volatile solvent in the sample were evenly distributed inside the test cell owing to the action of the fans.

After the thirty minutes time period has elapsed, the fan and the lamp of the photocatalytic reactor were turned on in order to start measuring the photocatalytic activity of the filter and its effect on an organic contaminant. The LED lamp (Eurolux) operated at 20 W. The fan (DC brushless QFR0812VH) operated at 4.5 V, such that air velocity was 1 m/sec and air flow rate across the photocatalytic reactor was 10 L/sec.

The measurement using the tiger detector was performed by connecting the tip of the detector (where the gaseous sample is drawn into the detector with the help of a built-in pump inside it) to the tap of the test chamber. The reading, which stabilizes after about 30 seconds, is the concentration in ppm of the tested organic gas inside the cell. Measurements were conducted periodically at one-hour intervals and continued until the concentration of the tested gas dropped below the detection limit owing the photocatalytic action of multiple combination of bismuth oxyhalides incorporated into the filter.

For comparison, the same experiment was carried out using titanium oxide-based photocatalytic device: “Air Oasis” photocatalytic air purifier, which was placed inside the 500 L test chamber.

It should be noted that the Tiger detector cannot tell which gas is in the cell, but calculates the concentration taking into consideration the response factor (RF) of the organic gas chosen, i.e. the concentration of the generated intermediates in the process is calculated using the RF of Toluene.

Results

The volatile organic solvents, the decomposition of which was tested (separately) in the study, were toluene and ethanol. Toluene samples of 4 microliters were used. Ethanol samples of 2 microliters were used.

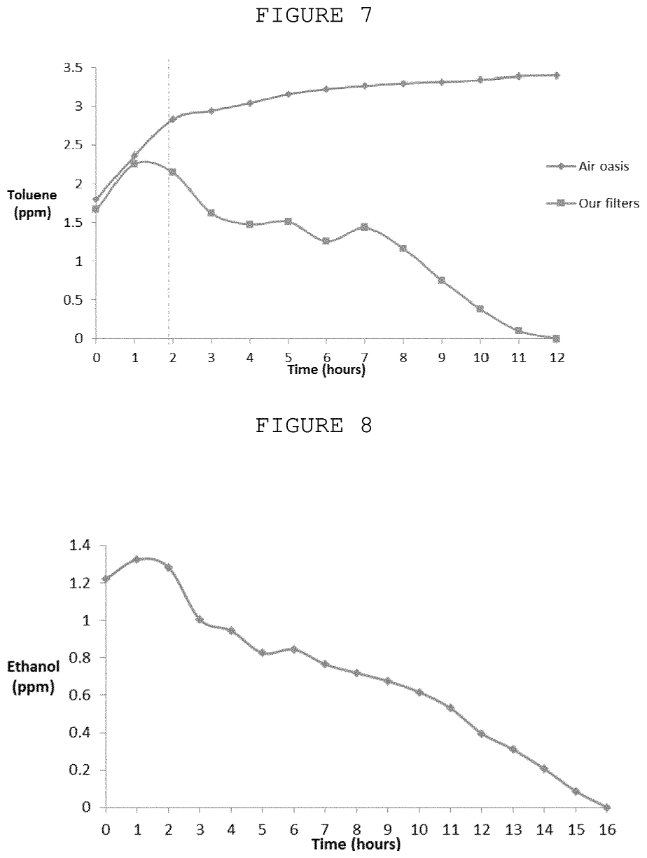

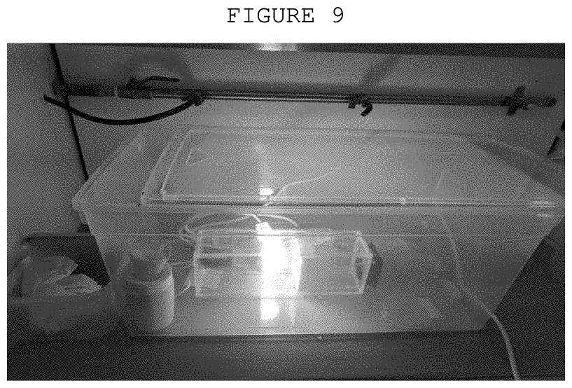

The results are shown in FIGS. 7 and 8, respectively, in the form of concentration (ppm) versus time plots. Results measured for the photocatalytic filter of the invention are indicated in squares. Results obtained for the comparative commercial photocatalytic unit are marked by rhombuses (Air Oasis).

Generally, a characteristic concentration versus time curve of photooxidation process of an organic contaminant by the action of a photocatalyst shows an initial increase of concentration, indicating the build-up of successively generated oxidation products. For example, in the case of toluene, the methyl attached to the aromatic ring provides the first oxidizable site: —CH₃→—CH₂OH→—CH(═O)→—COH(═O). Next, the aromatic ring is opened, followed by carbon chain breakage. An efficient photocatalyst should be able to proceed to decompose the oxidation products of the original contaminant, eventually reaching full mineralization, i.e., CO₂ and H₂O formation.

Turning now to the concentration versus time curves for toluene in FIG. 7, the results show that under the action of the photocatalytic filter of the invention, activated with visible light irradiation, the concentration of the organic matter increases over the first hour, in line with the explanation given above. The concentration then decreases gradually, dropping down to zero after 12 hours, indicating that toluene underwent full oxidation to carbon dioxide and water.

TiO₂-based commercial photocatalytic units tested in the study did not perform well:

Air Oasis shows steady increase in the organic matter from 1.8 ppm at time=0 to 3.4 ppm after 12 hours. This means that toluene was partially oxidized, but its (relatively stable) oxidation products haven't got fully oxidized to carbon dioxide and water by the action of Air Oasis.

Turning now to the concentration versus time curves for ethanol in FIG. 8, it is seen again the photocatalytic filter of the invention achieves complete mineralization of the organic matter after sixteen hours.

### Example 2

### Antimicrobial Activity of A1/B Combination Incorporated into Gypsum Model Filter

The goal of the study was to determine antimicrobial activity exerted by the combination of bismuth oxyhalides, to assess its potential benefit in air-disinfection, i.e., in eliminating bacteria from contaminated surfaces.

In the study reported in this Example, a combination of two active bismuth oxyhalides was formulated as an aqueous dispersion. The formulation was applied onto honeycomb-shaped, gypsum-made filter. The photocatalytic filter was mounted in a cell (photocatalytic reactor) equipped with visible light irradiation source to “switch on” the photocatalytic activity, and a fan to facilitate air flow across the cell. The experimental work was divided into two parts.

In part A, the photocatalytic cell was placed in a test chamber. Bacterial colonies (Salmonella typhi and Bacillus subtilis) grown on microslides were inserted into the test chamber, externally to the photocatalytic cell. Bacterial counts were taken periodically to assess the antimicrobial effect of the photocatalytic filter.

In part B, the photocatalytic cell was placed on shelf in a refrigerator. Bacterial colonies (Listeria monocytogenes ATCC) grown on microslides were put into the refrigerator (at two different locations). Bacterial counts were taken periodically to assess the antimicrobial effect of the photocatalytic filter.

Experimental Set-Up

1) A1+B Aqueous Formulation

10 g of component A (Bi⁽⁰⁾ doped-BiOCl_{0.80}Br_{0.20} of Preparation 1) and 30 g of component B (BiOCl_{0.20}Br_{0.80} of Preparation 4) were added to 100 ml water, to afford an aqueous dispersion of the two photocatalysts.

2) Photocatalytic Filter

A honeycomb-shaped filter, with the A1+B aqueous dispersion applied thereto, was prepared as described in Example 1.

3) Photocatalytic Reactor

The photocatalytic reactor is as described in Example 1 and shown in the photo appended in FIG. 5A.

4) Test Chamber

The test chamber consists of a 70-liter plastic container to accommodate the photocatalytic reactor. The test chamber was partially open to protect against uncontrolled air flow, but to allow air exchange at the same time.

Part A

Experimental Protocol

The photocatalytic reactor and contaminated glass slides were placed in the test chamber which was located inside a biological hood for safety reasons. The tests were performed in sterile conditions to prevent cross contamination. Two different microorganisms were chosen (Salmonella typhi and Bacillus subtilis), which represent the variety of bacteria and molds which are common airborne pollutants. The test chamber is shown in the photograph appended in FIG. 9. The photocatalytic reactor is in active mode (light source turned on). Contaminated microslides are located at the right side of the container.

The photocatalytic reactor started working when the LED light and the fan were turned on.

The contaminated glass slides were taken out for the counting of the microorganisms at predetermined intervals. They were transferred into test tubes where they were washed in order to start the counting process of the living microorganisms.

Results

Bacterial counts are tabulated below.

TABLE 1
Salmonella typhi

| Time [Hr] | CFU ((colony forming units) |
|---|---|
| 0 | 900,000 |
| 1 | 540,000 |
| 2 | 3,000 |
| 3 | 600 |

TABLE 2
Bacillus subtilis

| Time [Hr] | CFU ((colony forming units) |
|---|---|
| 0 | 1,200,000 |
| 1 | 680,000 |
| 2 | 4,400 |
| 4 | 960 |
| 6 | 500 |

The results demonstrate that the photocatalytic filter of the invention exerted antimicrobial activity, indicated by four-fold reduction of surface contamination. It is of note that the effect was achieved even though there was no direct contact between the photocatalytic filter and the bacterial colonies. Without wishing to be bound by theory, it is believed that the creation of oxidant species in the atmosphere inside the photocatalytic cell (i.e., decomposition of water molecules to produce active hydroxyl radicals) ultimately led to elimination of bacterial colonies. The location of the bacterially contaminated glass slides inside the test chamber had no effect on the microorganisms count results; no differences were found when the glass slides were located on the front or on the side of the test chamber. This suggests that uniform atmosphere was created inside the test chamber, in terms of the distribution of oxidant species.

Part B

Experimental Protocol

The photocatalytic reactor was placed on a shelf inside a refrigerator (T=2-8° C.). Listeria monocytogenes-contaminated microslides were placed in the refrigerator at two different locations:
Inside to the photocatalytic reactor: adjacent to the front wall of the photocatalytic reactor (i.e., the perforated wall opposite to the wall equipped with the fan).
Externally to the photocatalytic reactor: on a shelf in the refrigerator below the photocatalytic reactor.

Listeria monocytogenes-contaminated tube served as a control sample. The tube was covered with aluminum foil to cancel out any effect generated by the photocatalytic reactor. The control tube was placed on the shelf below the photocatalytic reactor.

At two time points after the beginning of the experiment (marked by switching on the photocatalytic reactor), i.e. after twelve and twenty-four hours of continuous operation of the photocatalytic reactor, the treated microslides were taken out of the refrigerator for viable counts. As to the control sample, viable count was performed only once, at the end of the twenty-four hours period. Details are as follows:

Starter of Listeria monocytogenes was grown over night. Serial dilutions were made to count the viable cells and determine initial concentration. Next, a volume of 0.2 ml of the starter was put on each of four microslides (an internally located pair, consisting of a first microslide for the t₁=12 h measurement and a second microslide for the t₂=24 h measurements+an externally located pair, consisting of a first microslide for the t₁=12 h measurement and a second microslide for the t₂=24 h measurements).

For the counting measurements, a slide was taken out from the refrigerator, inserted into 50 ml tube and washed by 2 ml of PBS buffer. The tube was vortexed and its content was transferred to a petri dish, into which SMA medium was poured to serve as the plate count agar. Viable counts were made on serially diluted samples incubated for forty-eight hours at 37° C.

Results

The results are tabulated in Table 3.

TABLE 3
Listeria monocytogenes

| | internally | | externally | | |
|---|---|---|---|---|---|
| | located samples | | located samples | | control |
| t = 0 h | 1.2 × 10⁸ | CFU/g | 1.2 × 10⁸ | CFU/g | 1.2 × 10⁸ CFU/g |
| T = 12 h | <100 | CFU/g | <100 | CFU/g | |
| T = 24 h | <10 | CFU/g | <10 | CFU/g | 1.0 × 10⁸ CFU/g |

The results indicate the strong antimicrobial effect exerted by the multiple combination of bismuth oxyhalides.

### Example 3

### Photooxidation Activity of A1/A3 Combination Embedded in Non-Woven Fabric Filter

The goal of the study was to assess the visible-light induced photooxidation generated by a combination of bismuth oxyhalides, when this combination is set in non-woven fabric filter medium.

In the study reported in this Example, a combination of two active bismuth oxyhalides was dispersed in ethanol. The binder-free formulation was sprayed onto non-woven fabrics. The fabrics were dried under ambient temperature, following which the bismuth oxyhalides-loaded non-woven fabric was mounted in a cell, equipped with visible light irradiation source (to “switch on” the photocatalytic activity) and a fan. Vapors of volatile organic solvents, generated in a sealed test chamber, were caused to flow through the cell and across the photocatalytic fabric filter. The concentration of the gaseous organic material was measured as function of time, to assess the ability of the photocatalytic non-woven fabric filter to decompose vapors of organic contaminants passing therethrough.

Experimental Set-Up

1) A1+A3 Ethanolic Formulation

350 mg of component A1 (Bi⁽⁰⁾ doped-BiOCl_{0.80}Br_{0.20} of Preparation 1) and 150 mg of component A3 (BiOBr)) were dispersed in 4 ml ethanol using a homogenizer (10,000 rpm).

2) Photocatalytic Filter

Different types of 1-2 mm thick non-woven fibers (some of which include activated carbon as an adsorbing agent) were cut into square-shaped pieces (10 cm×10 cm). A volume of 4 ml of the A1+A3 ethanolic dispersion was uniformly sprayed on each of the non-woven fabric pieces. The fabric pieces were dried by allowing the ethanol to evaporate at room temperature.

3) Photocatalytic Reactor

The photocatalytic reactor was the same 3 L rectangular cell described in Example 1 and shown in the photo appended in FIG. 5, but this time a bismuth oxyhalide-incorporated non-woven fiber piece served as the filter medium in place of the honeycomb-shaped gypsum body. The 10 cm×10 cm fiber piece was mounted in the photocatalytic reactor, 15 cm apart from the rear wall where the fan is located. The fiber piece was fit into a suitable frame made of Perspex.

4) Test Chamber

The test chamber is the same 500 L sealable cell, rectangular in shape, described in Example 1 and shown in FIG. 6. As mentioned above, the major elements of the test chamber include: a shelf mounted at the upper part of the test chamber, to hold a sample of a volatile organic solvent; a pair of fans to ensure distribution of the vaporized organic pollutant in the interior of cell and passage of the vapors through the photocatalytic reactor; a sealable door; and an external tap mounted in the center of one of its walls, to which the Tiger device is connected for VOC measurements; and humidity and temperature meters.

Experimental Protocol

The protocol was similar with the one described in Example 1 (pre-experiment ventilation of the test chamber, placement of petri dish with a volatile organic solvent on the shelf in the test chamber, evaporation of the volatile organic solvent to achieve uniform distribution of the gaseous contaminant in the test chamber, switching on the photocatalytic reactor (white LED lamp (Eurolux) 6500K, operated at 10 W; a fan (DC brushless QFR0812VH) operated at 4.5 V, such that air velocity was 1 m/sec and air flow rate across the photocatalytic reactor was 10 L/sec.

The measurements using the tiger detector were conducted periodically at 30 minutes intervals over a period of two hours.

Results

The volatile organic solvent, the decomposition of which was tested in the study, was toluene. Toluene samples of 2.13 microliters were used.

The results indicate that at the end of the two hours test period, the initial concentration of toluene (1 ppm) dropped significantly, with the photocatalytic filter achieving from 35 to 95% decomposition rates depending on the source of activated carbon and porosity of the fabric.

### Example 4

### Photooxidation Activity of A1/A3 Combination Embedded in Cabin Air Filter

The goal of the study was to assess the visible-light induced photooxidation generated by a combination of bismuth oxyhalides embedded in a cabin air filter. Such filters are loaded with activated carbon to capture particles, adsorb contaminants etc., to protect the heating ventilation and air conditioning system of the vehicle.

In the study reported in this Example, a combination of two active bismuth oxyhalides was dispersed in ethanol. The binder-free formulation was applied onto the filter. The bismuth oxyhalides-loaded pleated filter was mounted in a cell, equipped with visible light irradiation source to “switch on” the photocatalytic activity, and a fan. Vapors of volatile organic solvents, generated in a sealed test chamber, were caused to flow through the cell and across the photocatalytic cabin air filter. The concentration of the gaseous organic material was measured as function of time for more ten hours, to assess the ability of the photocatalytic cabin air filter to decompose vapors of organic contaminants passing therethrough.

Experimental Set-Up

1) A1+A3 Ethanolic Formulation

2 g of component A1 (Bi⁽⁰⁾ doped-BiOCl_{0.80}Br_{0.20} of Preparation 1) and 2 g of component A3 (BiOBr)) are dispersed in 25 ml ethanol using a homogenizer (10,000 rpm).

2) Photocatalytic Filter

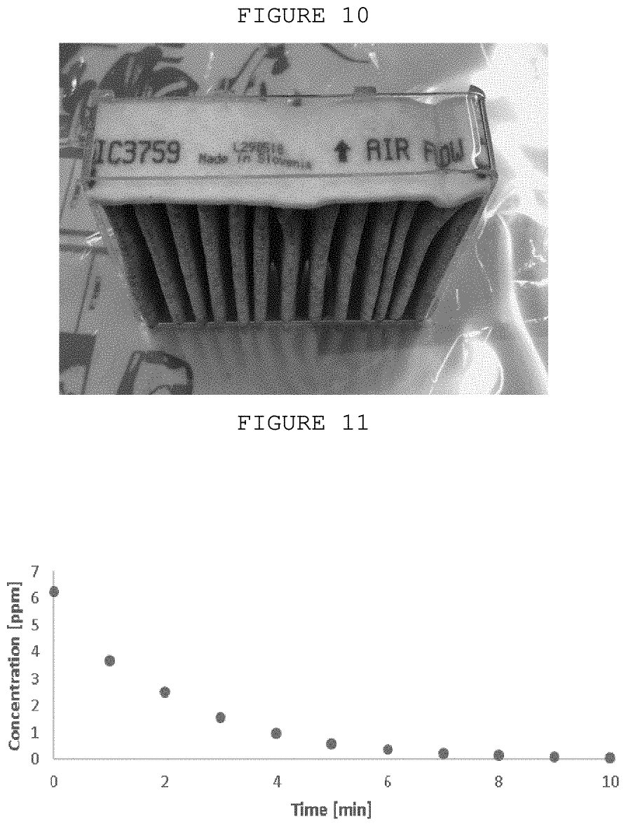

A volume of 25 ml of the A1+A3 ethanolic dispersion was uniformly sprayed on a 10 cm×10 cm×3 cm activated carbon-containing non-woven fabric filter. The filter was dried by allowing the ethanol to evaporate at room temperature. The pleated filter was fixed in a conventional frame (10 cm×10 cm open area), as shown in the photo appended in FIG. 10.

3) Photocatalytic Reactor

The photocatalytic reactor is the same 3 L rectangular cell described in Example 1 and shown in the photo appended in FIG. 5, but this time the bismuth oxyhalide-added, activated carbon-containing cabin air filter served as the filter medium in place of the honeycomb-shaped gypsum body. The cabin air filter was placed in the photocatalytic reactor, 12 cm apart from the rear wall where the fan is located.

4) Test Chamber

The test chamber is the same 500 L sealable cell, rectangular in shape, described in Examples 1 and 3, and shown in FIG. 6.

Experimental Protocol

The protocol was similar with the one described in Examples 1 and 3 (pre-experiment ventilation of the test chamber, placement of petri dish with a volatile organic solvent on the shelf in the test chamber, evaporation of the volatile organic solvent to achieve uniform distribution of the gaseous contaminant in the test chamber, switching on the photocatalytic reactor (white LED lamp (Eurolux) 6500K, operated at 20 W; a fan (DC brushless QFR0812VH) operated at 4.5 V, such that air velocity was 1 m/sec and air flow rate across the photocatalytic reactor was 10 L/sec.

The measurements using the tiger detector were conducted periodically every hour over a period of ten hours.

Results

The volatile organic solvent, the decomposition of which was tested in the study, was toluene. Toluene samples of 13.31 microliters were used.

The initial concentration of toluene in the test chamber was ˜6 ppm. A concentration versus time plot is shown in FIG. 11, indicating practically full mineralization of toluene at the end of the ten minutes test period.

### Example 5

### Testing the Action of Activated Carbon and Bismuth Oxyhalides: Adsorption and Photooxidation of Toluene and Formaldehyde

The goal of the study was to assess the ability of bismuth oxyhalides to aid activated carbon—the adsorbent used in filters—in eliminating volatile organic contaminants.

Experimental Set Up

The 3 L photocatalytic reactor described above, with its LED light source and fan positioned in the rear side, was used. However, a simplified configuration was adopted: in the comparative example, 500 mg of commercial activated carbon (Sigma-Aldrich Cat. 97876) were added to a Petri dish which was put inside the 3 L photocatalytic reactor. The photocatalytic reactor was placed in the 500 L test chamber.

In the experiment according to the invention, a powder blend consisting of:

200 mg of component A1 (Bi⁽⁰⁾ doped-BiOCl_{0.80}Br_{0.20} of Preparation 1);

50 mg of component A3 (BiOBr); and

250 mg of component B (BiOCl_{0.20}Br_{0.80} of Preparation 4) was added together with 500 mg of activated carbon to the petri dish in the photocatalytic reactor.

Experimental Protocol

The protocol was similar with the one described in Examples 1, 3 and 4 (pre-experiment ventilation of the test chamber, placement of petri dish with a volatile organic solvent on the shelf in the test chamber, evaporation of the volatile organic solvent to achieve uniform distribution of the gaseous contaminant in the test chamber, then switching on the photocatalytic reactor (white LED lamp (eurolux) 6500K, operated at 20 W; a fan (DC brushless QFR0812VH) operated at 4.5 V, such that air velocity was 1 m/sec and air flow rate across the photocatalytic reactor was 10 L/sec. Humidity % was ˜40%.

The measurements using the tiger detector were conducted periodically every thirty minutes over a period of twelve hours.

Results

Toluene sample of 8.52 microliters was added to the petri dish in the test chamber. The sample evaporated and the concentration of toluene inside the test chamber, before the experiment begun, was 4 ppm.

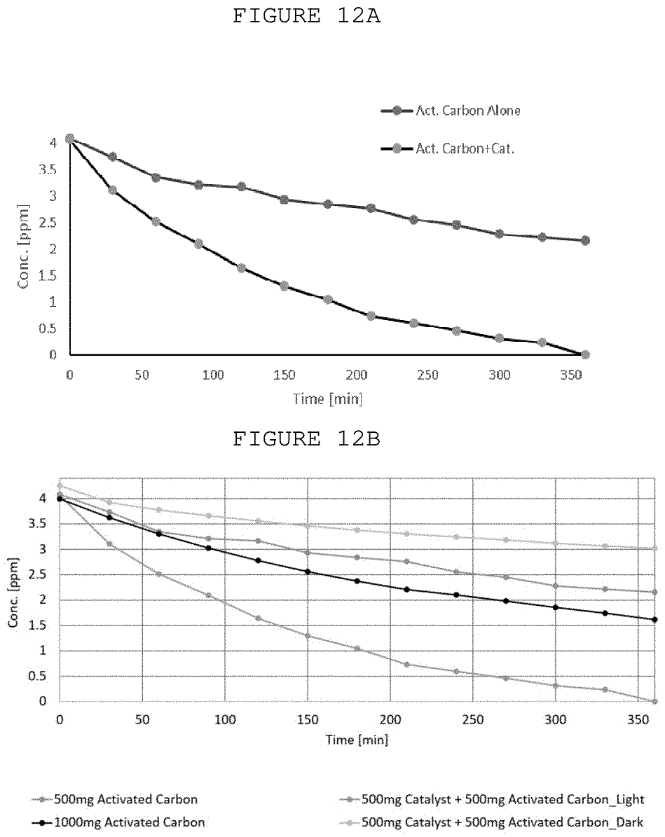

The elimination of toluene achieved with activated carbon alone (500 mg), and with a blend consisting of 500 mg activated carbon+500 mg of the [A1+A3]/B combination, was tested. The results are shown in FIG. 12A, in the form of concentration (ppm) versus time plots. It is seen that activated carbon alone cannot eliminate volatile organic contaminants effectively. The action of activated carbon/bismuth oxyhalides, combing adsorption and photooxidation, is much more effective. The results suggest that the photocatalyst, in addition to decomposing the pollutant, also attaches a self-cleaning functionality to the activated carbon adsorbent, thereby improving its performance.

FIG. 12B shows the results of FIG. 12A but adds two sets of data, collected with the same experimental set-up, using 1000 mg of activated carbon (the second closest curve to the abscissa) and a blend consisting of 500 mg activated carbon+500 mg of the [A1+A3]/B combination, but this time dark conditions (the uppermost curve). Notably, doubling the amount of the activated carbon (500 mg→1000 mg) achieves only slight improvement in the removal rate of toluene, as compared to the strong effect achieved by the blend of the invention. The results attest to the unique role of the photocatalyst in combination with activated carbon.

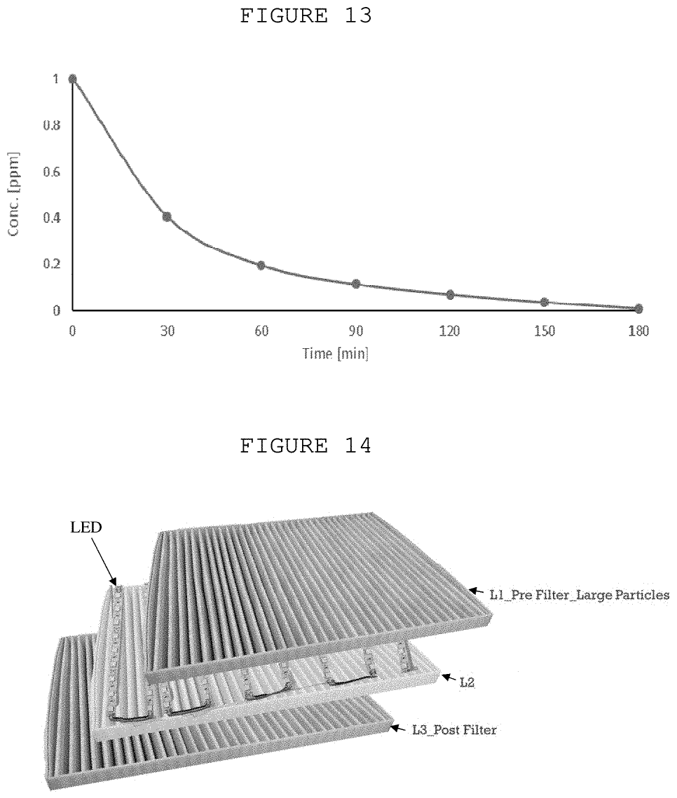

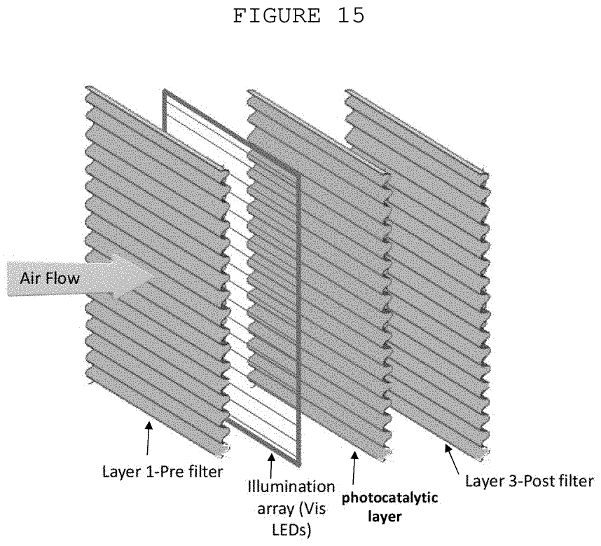

FIG. 13 is a concentration versus time curve illustrating the gradual elimination of formaldehyde (initial concentration in the test chamber 1 ppm) with the aid of activated carbon/bismuth oxyhalides blend. The formaldehyde photocatalytic oxidation process was monitored using a specific sensor produced by Graywolf.

### Example 6

### Antiviral Activity of A1 and A3 Photocatalysts

The goal of the study was to test antiviral activity of A1 and A3 of Preparation 1 and Preparation 3, respectively (both synthesized with the aid of quaternary ammonium halide salts) by neutralization of Vesicular Stomatitis Virus (VSV), which is an enveloped, negative-sense RNA virus with wide host range.

Experimental Set-Up

Virus stocks were prepared in monolayer cultures of HeLa cells growing in Dulbecco's modified Eagle's medium (DMEM). The DMEM was supplemented with 10% of fetal calf serum (FCS), 100 U/mL penicillin, 100 U/mL streptomycin, and 2 mM L-glutamine (Biological Industries, Beit Haemek, Israel).

Virus titration was held in 96 wells plates as follows: 50000 HeLa cells per well were plated 24 hours prior to infection. The cultures were infected with (50 μl) virus in decimal dilutions. Following an hour of absorption, the cultures were covered with 150 μl of DMEM supplemented with 2% FCS. The virus titer was determined 48 hours post infection. Cells were fixed with 1.7% formaldehyde for 30 minutes at room temperature, stained with 100 μl of 0.01% Crystal Violet, and then washed with tap water. The virus titer was determined by end-point dilution.

Experimental Protocol

Ten mg of each one of the A1 and A3 photocatalyst powders were mixed with the virus in 1 ml medium in transparent glass tubes. Each test was conducted for an hour, in room temperature, with continuous rotation, under controlled light conditions (LED lamp). Simultaneous control tests were conducted in tubes wrapped with aluminum foil to prevent light exposure (in the dark).

Photocatalyst co-incubated with the virus samples were collected at intervals of 10 minutes. Samples were centrifuged to separate the photocatalyst (a non-soluble powder) from the virus. Following centrifugation, each sample was serially diluted, and 50 μl of separated virus were added to the HeLa cell cultures growing in 96-well plate. After one hour of virus absorption, 150 μl of medium were added to each well, and the cells were incubated at 37° C. for 48 hours, when the virus titer was determined.

In parallel, the toxicity of the photocatalysts were assessed. “HeLa” cells cultures maintained as described above in DMEM media, were incubated with the catalysts in light and dark conditions. No cytotoxic effect was observed in the cell culture at all the concentration used for virus inactivation (10 mg/ml).

Results

The photocatalysts have shown a clear antiviral activity in lowering the virus concentration up to three orders within 30 minutes, and up to four orders within 50 minutes of virus incubation in light conditions. The results for the A1/A3 photocatalysts are tabulated below.

TABLE 4

| Light exposure | | Dark |
|---|---|---|
| (min) | Light | (control) |
| 0 | 10⁴ | 10⁴ |
| 10 | 10² | 10⁴ |
| 20 | 10² | 10³ |
| 30 | <10 | 10³ |
| 40 | <10 | 10³ |
| 50 | <10 | 10⁴ |
| 60 | <10 | 10⁴ |

### Examples 7A-7D

### Photooxidation Activity of A1 Photocatalyst: Decomposition of Volatile Organic Compound

A series of experiments were conducted to test the action of Bi⁽⁰⁾ doped-BiOCl_{0.80}Br_{0.20} of Preparation 1 on volatile organic compounds (VOCs).

In its most general form, the experimental set-up consisted of the previously described test chamber (see FIG. 6), in the form of 500 L Perspex cell, accommodating a 30 cm×10 cm×10 cm photoreactor (the design of photoreactor was modified compared to that used in previous examples, as explained below). Toluene was the VOC of choice the experiments; toluene was added to a petri dish that was placed on a shelf mounted in the upper section of the test chamber. A pair of fans installed in the test chamber as previously described in reference to FIG. 6 enabled evaporation of toluene and its uniform distribution in the interior test chamber, such that it can reach the photocatalytic reactor. Variation in toluene concentration in the test chamber was detected with Tiger VOC detector (from Ion Science).

As for the photoreactor, reference is made to the design shown in FIG. 5B. A fan (5C) (San Ace 80 model name: 109P0812M601) is installed in one of the square-shaped sides of the photoreactor (2) to move air from the test chamber into the reactor. LED strips were mounted inside the photoreactor, in place of the previously used LED lamp. A total of five LED strips (7) were affixed to a frame, in parallel to each other, separated by equal distances of 2 cm. The frame itself can be installed in the photoreactor at two different positions:

1) perpendicularly to the longitudinal axis of the photoreactor, such that the LED strips are positioned vertically (e.g., at distance of 10 cm from the fan; the frame is movable and can be repositioned along the length of the photoreactor); and

2) the frame can be suspended from the ceiling of the photoreactor, such that the LED strips (7) are positioned horizontally, facing the floor of the photoreactor.

The inner walls of the photoreactor are partially coated with mirrors to deflect the light beams in the direction of the tested sample.

The photocatalyst tested was placed inside the photoreactor in various ways, i.e., embedded in, or applied onto the surface of substrates designed to allow flow-through of moving air. For example, numeral (8) in the appended drawings indicates a flow-through support coated with the photocatalysts. But other modes of using the photocatalysts were tested, as shown in each of the experiments 7A-7D.

7A (Direct Action of Photocatalyst Powder):

The powdery photocatalyst Bi⁽⁰⁾ doped-BiOCl_{0.80}Br_{0.20} (2 g) was added to a petri dish placed in the interior of the photoreactor, about 20 cm from fan. The LED array was mounted above the petri dish, i.e., the LED strips (7) are positioned horizontally, illuminating the powder that rests on the floor of the photoreactor.

Toluene was added to a petri dish in the test chamber and was evaporated to reach toluene concentration of 3 ppm in the sealed test chamber (i.e., initial VOC level).

Then the fan of the photoreactor was turned on (operating at 50% of its maximal intensity, achieving incoming air stream of 0.5 m/s velocity). The LED illumination was switched on (approximately 15 W power), to induce the photocatalytic action of the Bi⁽⁰⁾ doped-BiOCl_{0.80}Br_{0.20} powder.

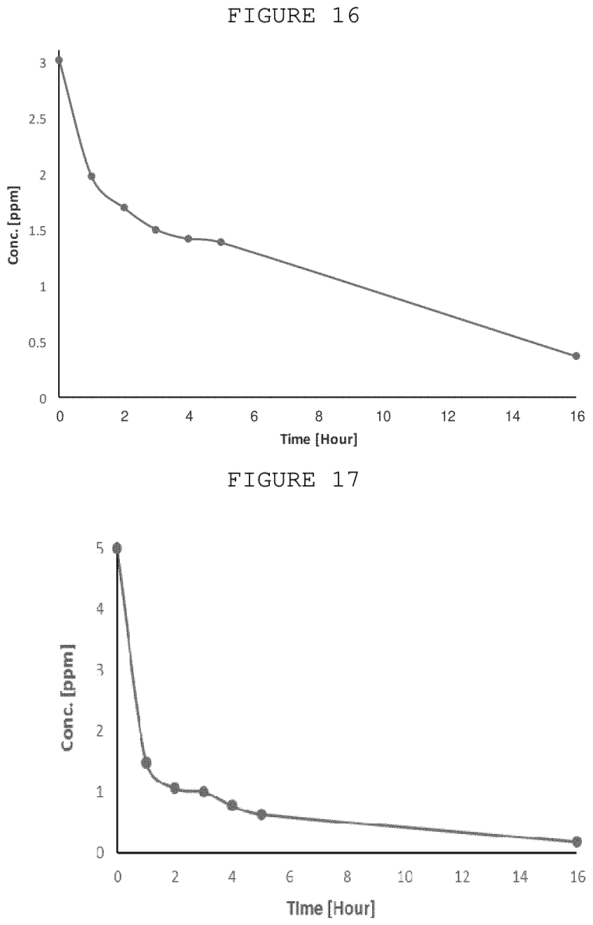

The results are shown in FIG. 16, where toluene concentration is plotted versus time. The results demonstrate degradation of toluene with the passage of time (toluene concentration dropped by 50%˜five hours after the experiment had begun).

7B (Photocatalyst Embedded in a Flow-Through Gypsum Substrate:

The photocatalyst was incorporated into a gypsum filter by the following method. Gypsum powder (60 g), activated carbon (1.5 g; Sigma Aldrich 31616) and silica (1 g; Sigma Aldrich 60760) were added to double distilled water (50 ml) which contained the Bi⁽⁰⁾ doped-BiOCl_{0.80}Br_{0.20} photocatalyst (10 g). Following the initial mixing, the resulting photocatalytic gypsum formulation was poured over a thin silicon-based template as previously described and left for a final drying over 2 hours. The hardened 10 cm×10 cm×1.5 cm photocatalyst-added honeycomb shaped gypsum block, with an array of open cells extending through the block having hexagonal cross-section, to enable passage of air across the gypsum block, was ready for use. The photocatalytic gypsum was installed inside the photoreactor to occupy the square cross section (10 cm×10 cm) of the photoreactor.

Toluene was added to a petri dish in the test chamber and was evaporated to reach toluene concentration of 5 ppm in the sealed test chamber (i.e., initial VOC level).

Then the fan of the photoreactor was turned on (operating at 70% of its maximal intensity). The LED illumination was switched on (full power), to induce the photocatalytic action of the Bi⁽⁰⁾ doped-BiOCl_{0.80}Br_{0.20} powder embedded in the gypsum filter.

The results are shown in FIG. 17 in the form of concentration versus time curve, demonstrating rapid degradation of toluene:toluene concentration dropped by 90%, only two hours after the experiment had begun.

7C (Photocatalyst Applied on a Flow-Through Metal Substrate):

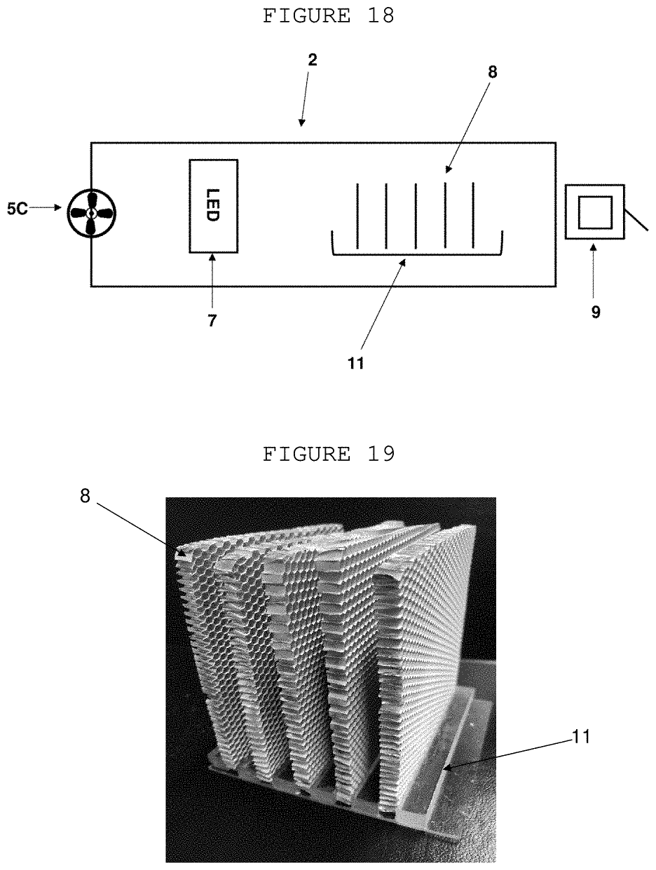

The experimental set-up is shown in FIG. 18, schematically illustrating a side view of the photoreactor (2). A fan (5C) was installed in one face of the photoreactor and the array of LED strips (7) was positioned vertically as previously explained. The change is seen in the addition of a white LED lamp (9), positioned outside the photoreactor, about 5 cm apart from the face of the photoreactor opposite the fan, for illuminating an array of tested samples indicated by numeral (8).

A photograph of the array of tested samples is shown in FIG. 19. Each member of the array has a structure of a honeycomb, i.e., hollow cells formed between thin (1 mm) aluminum walls. The side of the hexagonal cross-section of the hollow cell is 3 mm. The aluminum-made honeycomb corresponds in size and shape to the dimensions of the photoreactor such that it can fitted inside the photoreactor in a transverse position, to force air moving in the photoreactor to pass through the hollow cells of the aluminum-made honeycomb. Each aluminum-made honeycomb is 6 mm thick. As pointed out above, a total of five aluminum-made honeycombs was used, positioned in parallel to each along the longitudinal axis of the photoreactor.

Adjacent aluminum-made honeycombs are spaced 1 cm apart. As shown in FIG. 19, the aluminum-made honeycombs are joined to a base (11) such that the entire array can be inserted into, and taken out from, the photoreactor.

To apply the Bi⁽⁰⁾ doped-BiOCl_{0.80}Br_{0.20} powder onto the thin aluminum walls, each aluminum-made honeycomb was treated with a sprayable glue (suitable glues are available commercially; the binding agent is dispersed in organic solvent(s); sometimes a diluent is added just before application). After the volatile organic component has evaporated, the Bi⁽⁰⁾ doped-BiOCl_{0.80}Br_{0.20} powder was applied onto the glue-coated aluminum walls, by spraying an isopropanol dispersion of the photocatalyst (˜1 g powder in 10 cc IPA), to create a thin layer of the photocatalysts onto the walls of the hollow cells of the structure. The amount of photocatalyst loaded onto each aluminum-made honeycomb, with the geometric features set out above, was 0.7 g. The array consisting of the five aluminum-made honeycombs was placed inside the photoreactor.

Toluene was added to a petri dish in the test chamber and was evaporated to reach toluene concentration of 3 ppm in the sealed test chamber (i.e., initial VOC level). Then the fan of the photoreactor was turned on (operating at 75% of its maximal intensity). The LED illumination was switched on (full power), and also the externally positioned white LED projector (10 W), to trigger the photocatalytic action of the Bi⁽⁰⁾ doped-BiOCl_{0.80}Br_{0.20} powder applied onto the walls of the aluminum-made honeycombs.

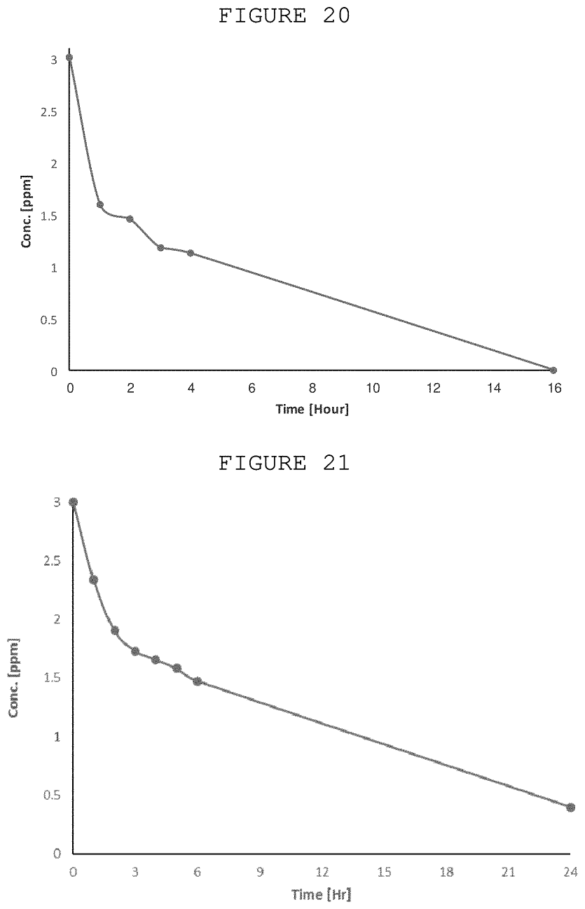

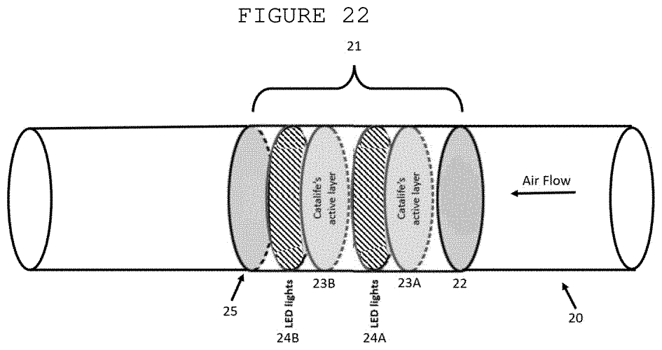

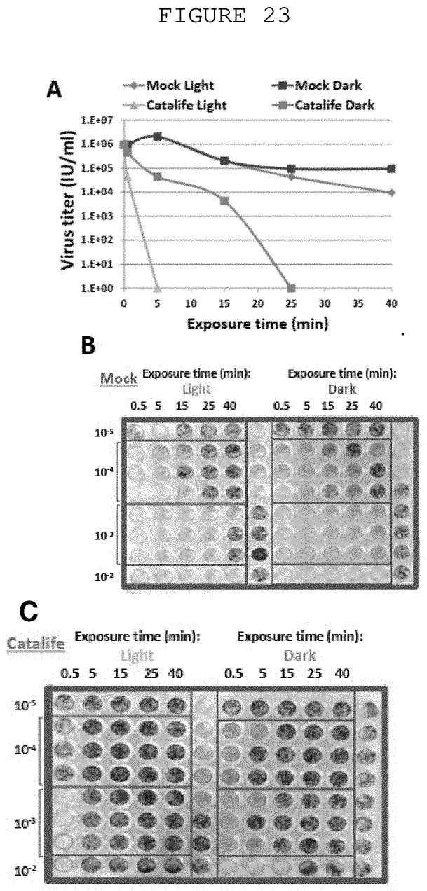

The results are shown in FIG. 20, indicating a rapid decrease in the VOC level in the test chamber: toluene concentration was reduced by half after just one hour, achieving full degradation at the end of the experiment.

7D (Photocatalyst Applied on a Flow-Through Fabric Substrate):

This time the photocatalyst was added to an elastic woven fabric made of polyester. Four pieces (10 cm×10 cm in size) fabric were used; each was uniformly coated with 0.7 g of the photocatalyst by the procedure set out above (i.e., first coating the fabric with a glue provided in an organic carrier, (using a spray gun), allowing the volatile carrier to evaporate and then applying an isopropanol dispersion of the Bi⁽⁰⁾ doped-BiOCl_{0.80}Br_{0.20} photocatalyst on one face of the fabric, by brushing or spraying. The four square-shaped pieces of fabric, to which the photocatalyst was added, were affixed to a frame inside the photoreactor.

Toluene was added to a petri dish in the test chamber and was evaporated to reach toluene concentration of 3 ppm in the sealed test chamber (i.e., initial VOC level). Then the fan of the photoreactor was turned on (operating at 75% of its maximal power). The LED illumination was switched on (full power).

The good removal rate of the VOC by the action of the photocatalyst incorporated into the flow-through fabric is demonstrated by a concentration versus time plot shown in FIG. 21.

### Example 8

### Inactivation of SARS-CoV-2 Virus by Bi⁽⁰⁾ Doped-BiOCl_{0.80}Br_{0.20}

The anti-viral activity of Bi⁽⁰⁾ doped-BiOCl_{0.80}Br_{0.20} photocatalytic powder against SARS-CoV-2 virus was examined in infected cells.

Experimental Set-Up

Vero-E6 cells were grown in DMEM medium supplemented with penicillin (100 U/mL), streptomycin (100 U/mL), L-glutamine (2 mM) and FCS (10%). One day before infection, cells were seeded at 10⁴ cells/well in 96-well plates. After infection cells were grown in DMEM supplied with 1% FCS.

SARS-Related Coronavirus 2 Isolate USA-WA1/2020 (BEI Resources, Cat. number NR-52281) was used for production of viral stock for the experiment. Initial dilution of 1:100 was prepared for a working stock and for incubation with the photocatalyst compound powder.

Experimental Protocol

Bi⁽⁰⁾ doped-BiOCl_{0.80}Br_{0.20} photocatalyst powder (25 mg) was mixed with 1 ml of the working SARS-CoV-2 stock in sterilized glass vials and incubated in visible light (10 W Daylight LED lamp) or dark conditions for time intervals of 0.5, 5, 15, 25 and 40 minutes. After the incubation, the virus-compound mixtures were transferred to 1.5 ml tubes (Eppendorf) and briefly span down (by centrifugation at 2500 rpm, 3 minutes, 4° C.) to separate the virus from the non-soluble powder. The supernatants were subjected to serial dilutions in DMEM medium (without FCS) and 50 μl of the diluted viruses were added to the Vero-E6 cells for infecting thereof (by absorption). The experiment was performed at triplicates. After 1 hour of incubation, 150 μl of fresh medium were added to the infected cells (DMEM, 1% FCS final concentration). Cells were incubated for further 48 hours in an incubator (CO₂ 5%, 37° C.). Mock virus samples (control) were incubated in glass vials without any powder and were used to infect cells as detailed above. The experiments conducted with infective virus were carried out in the HUJI BSL3 laboratory (The Hebrew University—Hadassah Medical School, Ein Kerem), strictly according to the NIH safety Biosafety level 3 guidelines for work with infectious agents.

Calculation of the virus titer (end-point dilution assay) was performed as follows. After 48 hours of incubation (namely, post infection), cells were fixed with 4% Formaldehyde for 30 minutes, then washed with phosphate-buffered saline (PBS) and stained with Crystal Violet (0.05%) for 10 minutes. After removing the stain, the wells were examined for the Cytopathic Effect (CPE), namely structural changes in the host cells resulting from viral infection, while wells empty of cells were detected as CPE-positive and blue-stained monolayers were detected as CPE-negative. Calculation of the viruses' titers in each sample was performed according to the modified Ramakrishnan Formula (DOI:10.13140/2.1.4777.1209).

Results:

As shown in FIG. 23, the Bi⁽⁰⁾ doped-BiOCl_{0.80}Br_{0.20} photocatalyst successfully reduced the virus infectivity, from 10⁶ IU/ml to actually zero. It is noteworthy that FIG. 23A presents the infectivity at 5 minutes in visible light and at 25 minutes at dark conditions as 1 IU/ml, while in fact, no CPE was observed at the highest virus concentration, as evident from the results shown in FIG. 23C (this was done in order to plot the data at the logarithmic scale (y-axes)). In other words, 5 minutes of incubation under visible light was sufficient to inactivate the virus, while full inactivation was achieved also in dark conditions, after 25 minutes, indicating a possible antiviral activity over dark conditions due to the presence of Bi(0) nanoparticles in the compound doped-heterojunctioned material. It is also noteworthy that SARS-CoV-2 virus is apparently sensitive to light, since the incubation under light conditions without any compound decreases the virus titer by one order of magnitude (log 10) after 40 min of exposure (FIG. 23B).

### SUMMARY

The Bi⁽⁰⁾ doped-BiOCl_{0.80}Br_{0.20} compound (at 25 mg/ml) completely inactivated the SARS-CoV-2 virus after 5 minutes of light exposure, when the initial titer was as high as 10⁶ IU/ml.

1) A combination comprising at least two bismuth oxyhalide compounds selected from Groups A1, A2, A3 and B, wherein:
Group A1 includes Bi⁽⁰⁾ doped-bismuth oxyhalides;
Group A2 includes bismuth oxyhalides of the formula BiOCl_{y}Br_{1-y}, with y≥0.5;
Group A3 includes single halide bismuth oxyhalides; and
Group B includes bismuth oxyhalides of the formula BiOCl_{y}Br_{1-y}, with y<0.5.

2) A combination according to claim 1, wherein:
Group A1 includes Bi⁽⁰⁾ doped-BiOCl, Bi⁽⁰⁾ doped-BiOBr and Bi⁽⁰⁾doped-BiOCl_{y}Br_{1-y} with 0.6≤y≤0.95;
Group A2 includes BiOCl_{y}Br_{1-y} with 0.6≤y≤0.95;
Group A3 includes BiOHal wherein Hal is chloride or bromide; Group B includes BiOCl_{y}Br_{1-y} with 0.1≤y≤0.4.

3) A combination according to claim 1, comprising Group A1 compound.

4) A combination according to claim 3, comprising Group A3 compound and/or Group B bismuth oxyhalide.

5) A combination according to claim 4, comprising:
Group A1 compound, which is Bi⁽⁰⁾doped-BiOCl_{y}Br_{1-y} [0.7≤y≤0.95]; and at least one of:
Group A3 compound, which is BiOBr, in the form of flower-like microspheres;
Group B compound, which is BiOCl_{y}Br_{1-y} [0.1≤y≤0.4] in the form of plates or flower-like microspheres;
wherein the combination is a photooxidant, antibacterial and antiviral.

6) A composition comprising the bismuth oxyhalides combination of claim 1 in water, a volatile organic solvent or a mixture thereof.

7) A filter medium comprising a combination of bismuth oxyhalides as defined in claim 1, added to a flow-through support.

8) A filter medium according to claim 7, wherein the flow-through support is made of nonwoven or woven fabric.

9) A filter medium according to claim 7, where the flow-through support is in the form hollow cells defined by thin gypsum walls.

10) A filter medium according to claim 7, where the flow-through support is in the form hollow cells defined by thin metal walls.

11) A blend comprising activated carbon and combination of bismuth oxyhalides as defined in claim 1.

12) A multistage filter comprising VOC-decomposing and/or bacteria-eliminating and/or virus eliminating filter medium in the form of photocatalyst added to a flow-through support, placed downstream to a prefilter, with light source positioned between said prefilter and said photocatalyst.

13) A multistage filter according to claim 12, wherein the photocatalyst is Bi⁽⁰⁾ doped-bismuth oxyhalide, to eliminate viruses.

14) A multistage filter according to claim 13, wherein the Bi⁽⁰⁾ doped-bismuth oxyhalide is Bi⁽⁰⁾doped-BiOCl_{y}Br_{1-y} (0.7≤y≤0.95).

15) A multistage filter according to claim 12, wherein the photocatalyst added to the flow-through support comprises a combination of bismuth oxyhalides, optionally in admixture with activated carbon, the combination comprising at least two bismuth oxyhalide compounds selected from Groups A1, A2, A3 and B, wherein:
Group A1 includes Bi⁽⁰⁾ doped-bismuth oxyhalides;
Group A2 includes bismuth oxyhalides of the formula BiOCl_{y}Br_{1-y}, with y≥0.5;
Group A3 includes single halide bismuth oxyhalides; and
Group B includes bismuth oxyhalides of the formula BiOCl_{y}Br_{1-y}, with y<0.5.

16) A multistage filter according to claim 12, comprising bismuth oxyhalides added to a flow-through support, optionally in admixture with activated carbon, wherein said flow-through support is disposed between a pre-filter layer and a post-filter layer, wherein said pre-filter and post-filter layers are particulate-trapping layers, and wherein the light source consists of a plurality of LED lamps illuminating the photocatalyst.

17) A multistage filter according to claim 16, which is a cabin air filter.

18) A multistage filter according to claim 16, which is an air conditioner filter installed in the air flowing area of an air tube or an air conditioning system.

19) A transparent photocatalytic cell, having an air inlet and an air outlet, comprising:
bismuth oxyhalides-added filter medium mounted inside the cell;
means for drawing outside air stream or circulated air stream into the cell and forcing said air stream across said filter medium,
wherein the bismuth oxyhalides photocatalyst is activatable by daylight entering the cell or visible light source positioned to illuminate said photocatalyst.

20) A method for reducing biological load on surfaces, comprising forcing air in a space where the surfaces are placed to pass across a filter medium having a combination of bismuth oxyhalides applied on flow-through support, wherein said bismuth oxyhalides include bromide-predominant mixed halide of the formula BiOCl_{y}Br_{1-y}, with y<0.5, said bismuth oxyhalides being illuminated by visible light, to charge the air with oxidant species and reduce the level of microorganism on said surfaces without the direct application of oxidant species onto said surfaces.
